(19) United States
(12) Patent Application Publication
(10) Pub. No.: US 2021/0353710 A1
(43) Pub. Date: Nov. 18, 2021

(54) GLP-1R AGONIST AND METHODS OF TREATMENT

(71) Applicant: The Johns Hopkins University, Baltimore, MD (US)

(72) Inventors: Elia Duh, Baltimore, MD (US); Zhenhua Xu, Baltimore, MD (US); Lingli Zhou, Baltimore, MD (US); Seulki Lee, Baltimore, MD (US); Yumin Oh, Baltimore, MD (US)

(21) Appl. No.: 17/194,177

(22) Filed: Mar. 5, 2021

Related U.S. Application Data
(60) Provisional application No. 62/985,811, filed on Mar. 5, 2020.

(51) Int. Cl. A61K 38/16; A61K 47/60; A61P 27/02; A61K 45/06

(52) U.S. Cl. CPC A61K 38/16; A61K 45/06; A61P 27/02; A61K 47/60

(57) ABSTRACT

In one aspect, GLP-1r agonists including long-acting PEGylated exenatide (NLY01), exenatide, liraglutide, exenatide LAR (long acting), taspoglutide, albiglutide, dulaglutide, lixisenatide, semaglutide, LY2189265 are used to treat ocular ischemic, inflammatory, angiogenic, and/or degenerative conditions.

### RELATED APPLICATIONS

This application claims the benefit of priority under 35 U.S.C. § 119(e) to U.S. Provisional Application No. 62/985,811, filed Mar. 5, 2020, the entire contents of which is incorporated herein by reference in its entirety.

### STATEMENT REGARDING FEDERALLY SPONSORED RESEARCH

This invention was made with government support under grant numbers EY022383 and EY022683 awarded by the National Institutes of Health. The government has certain rights in this invention.

### FIELD OF THE DISCLOSURE

The present disclosure relates in general to methods of treating ocular diseases, disorders or injury. In particular aspects, GLP-1r agonists including long-acting PEGylated exenatide (NLY01), exenatide, liraglutide, exenatide LAR (long acting), taspoglutide, albiglutide, dulaglutide, lixisenatide, semaglutide, LY2189265 are used to treat ocular ischemic, inflammatory, angiogenic, and/or degenerative conditions.

### BACKGROUND

Ophthalmologic diseases affect millions of people around the world, particularly older adults. Many disorders are known to affect the eye, including macular degeneration, also known as age-related macular degeneration (AMD or ARMD), juvenile macular degeneration, retinal degeneration, glaucoma, retinal dystrophy, Doyne honeycomb retinal dystrophy, Stargardt disease, light induced retinal damage, uveitis, scleritis, ocular sarcoidosis, optic neuritis, cone-rod dystrophy, macular edema, diabetic retinopathy, diabetic macular edema, branch and central retinal vein occlusion, corneal ulcer, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, Retinoblastoma, Usher syndrome, Behcet's disease, Achromatopsia 2, Acute posteriormultifocal placoid pigment epitheliopathy (APMPPE), Acute zonal occult outer retinopathy (AZOOR), Adult-onset vitelliform macular dystrophy (AVMD), Ocular albinism with late-onset sensorineural deafness (OASD), Alstrom syndrome, Anterior ischemic optic neuropathy, corneal amyloidosis, Gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, Birdshot chorioretinopathy, Blue cone monochromatism, central areolar choroidal dystrophy, Choroideremia, Coats disease, Iridocornealendothelial (ICE) syndrome, Avellino type corneal dystrophy, Schnyder corneal dystrophy, Thiel-Behnke corneal dystrophy, Eales disease, Epithelial basement membrane corneal dystrophy, Fish-eye disease, Fuchs endothelial corneal dystrophy, Goldmann-Favre syndrome, Juvenile retinoschisis, late-onset retinal degeneration, Leber congenital amaurosis, retinitis pigmentosa, Peters anomaly, Punctate inner choroidopathy, Senior Loken syndrome, Snowflake vitreoretinal degeneration, Usher syndrome, Visual snow syndrome, Wagner syndrome, and other inherited retinal degenerations. Each of these can lead to visual loss or complete blindness.

### SUMMARY

In a first aspect, long-acting PEGylated exenatide (NLY01), exenatide, liraglutide, exenatide LAR (long acting), taspoglutide, albiglutide, dulaglutide, lixisenatide, semaglutide, LY2189265 are used to treat ocular ischemic, inflammatory, angiogenic, and degenerative conditions.

Accordingly, embodiments are directed to the treatment of ocular diseases and disorders, related to, for example, inflammation, autoimmune diseases, ischemia, neovascularization, neurodegeneration and the like. Pharmaceutical compositions for treating ocular diseases or disorders comprise therapeutically effective amounts of at least one agent, wherein the at least one agent comprises long-acting PEGylated exenatide (NLY01), exenatide, liraglutide, exenatide LAR (long acting), taspoglutide, albiglutide, dulaglutide, lixisenatide, semaglutide, LY2189265 or analogs thereof.

In other aspects, we have found glucagon-like peptide 1 receptor (GLP-1r) agonists, for example, long-acting PEGylated exenatide GLP-1r agonist, when administered either systemically or locally delivered, despite having a large molecular weight, can have significant protective and disease-modifying effects in ocular disease, including ischemic, inflammatory, angiogenic, and neurodegenerative disease conditions.

Without being bound by any theory, agents, such as, long-acting PEGylated exenatide (NLY01), exenatide, liraglutide, exenatide LAR (long acting), taspoglutide, albiglutide, dulaglutide, lixisenatide, semaglutide, LY2189265 may function via GLP-1r dependent or GLP-1r independent mechanisms in the treatment of such ocular diseases or disorders.

In certain embodiments, a method of treating an ocular neuroinflammatory disease, an ocular disease or disorder, an ocular autoimmune disease or ocular injury comprising administering to a subject a pharmaceutical composition, wherein the pharmaceutical composition comprises a therapeutically effective amount of at least one glucagon-like peptide 1 receptor (GLP-1r) agonist or analogs thereof, thereby treating an ocular neuroinflammatory disease, an ocular disease or disorder, an ocular autoimmune disease or ocular injury.

In certain embodiments, a method of treating an ocular neuroinflammatory disease, an ocular disease or disorder, an ocular autoimmune disease or ocular injury comprising administering to a subject a pharmaceutical composition, wherein the pharmaceutical composition comprises a therapeutically effective amount of at least one agent, wherein the agent comprises long-acting PEGylated exenatide (NLY01), exenatide, liraglutide, exenatide LAR (long acting), taspoglutide, albiglutide, dulaglutide, lixisenatide, semaglutide, LY2189265 or analogs thereof, thereby treating an ocular neuroinflammatory disease, an ocular disease or disorder, an ocular autoimmune disease or ocular injury.

In certain embodiments, the ocular neuroinflammatory disease, the ocular disease or disorder, the ocular autoimmune disease or ocular injury comprises: neuromyelitis optica spectrum disorder, optic neuritis, acute disseminated encephalomyelitis (ADEM), autoimmune uveitis, intraocular inflammation, uveitis, uveoretinitis, proliferative vitreoretinopathy, proliferative diabetic retinopathy (PDR), macular edema, non-proliferative diabetic retinopathy, branch and central retinal vein occlusion, dry and wet age-related macular degeneration, ocular neovascularization, retinopathy of prematurity (ROP), a retinal vein occlusion, ocular ischemic syndrome, acute and chronic glaucoma, neovascular glaucoma, a retinal hemangioma, Coates' disease, familial exudative vitreoretinopathy (FEVR), Norrie disease, macular degeneration, diabetic retinopathy, retinitis pigmentosa, cone dystrophy geographic atrophy, detachment ischemia, optic neuropathies including anterior ischemic optic neuropathy and optic nerve neuritis, ocular cancer, glaucoma, retinal trauma, physical trauma to the optic nerve and surrounding tissues, or retinal nerve damage.

In certain embodiments, a method of reducing and/or inhibiting vascular degeneration and/or pathological neovascularization and/or promoting reparative angiogenesis, comprising administering to a subject a pharmaceutical composition, wherein the pharmaceutical composition comprises a therapeutically effective amount of at least one glucagon-like peptide 1 receptor (GLP-1r) agonist or analogs thereof, thereby reducing and/or inhibiting vascular degeneration and/or pathological neovascularization.

In certain embodiments, a method of reducing and/or inhibiting vascular degeneration and/or pathological neovascularization and/or promoting reparative angiogenesis, comprising administering to a subject a pharmaceutical composition, wherein the pharmaceutical composition comprises a therapeutically effective amount of at least one agent, wherein the agent comprises long-acting PEGylated exenatide (NLY01), exenatide, liraglutide, exenatide LAR (long acting), taspoglutide, albiglutide, dulaglutide, lixisenatide, semaglutide, LY2189265 or analogs thereof, thereby reducing and inhibiting vascular degeneration and/or pathological neovascularization.

In certain embodiments, a method of modulating microglia activation in the eye of a subject, comprising administering to a subject a pharmaceutical composition, wherein the pharmaceutical composition comprises a therapeutically effective amount of at least one glucagon-like peptide 1 receptor (GLP-1r) agonist or analogs thereof.

In certain embodiments, a method of modulating microglia activation in the eye of a subject, comprising administering to a subject a pharmaceutical composition, wherein the pharmaceutical composition comprises a therapeutically effective amount of at least one agent, wherein the agent comprises long-acting PEGylated exenatide (NLY01), exenatide, liraglutide, exenatide LAR (long acting), taspoglutide, albiglutide, dulaglutide, lixisenatide, semaglutide, LY2189265 or analogs thereof.

In certain embodiments, the GLP-1r agonist comprises PEGylated exenatide (NLY01), exenatide, liraglutide, exenatide LAR (long acting), taspoglutide, albiglutide, albiglutide, dulaglutide, lixisenatide, semaglutide, LY2189265 or combinations thereof. In certain embodiments, the PEGylated exenatide (NLY01), exenatide, liraglutide, exenatide LAR (long acting), taspoglutide, albiglutide, albiglutide, dulaglutide, lixisenatide, semaglutide, LY2189265 or combinations thereof, act via GLP-1r dependent and/or GLP-1r independent mechanisms.

In certain embodiments the GLP-1r agonist or analogs thereof, is a long-lasting agonist or analog thereof. In certain embodiments, the GLP-1r agonist or analogs thereof is NLY01. In certain embodiments, the long-acting GLP-1r agonist comprises a PEGylated GLP-1 analog or derivative thereof.

In certain embodiments, the PEGylated exenatide (NLY01), functions via GLP-1r dependent and/or GLP-1r independent mechanisms.

In certain embodiments, the PEG moiety or derivative is selected from the group consisting of linear PEG, branched PEG, Star PEG, Comb PEG, dendrimeric PEG, PEG succinimidylpropionate, PEG N-hydroxysuccinimide, PEG propionaldehyde, PEG maleimide, linear methoxypoly(ethylene glycol) (mPEG), branched mPEG, Star mPEG, Comb mPEG, dendrimeric mPEG, mPEG succinimidylpropionate, mPEG N-hydroxysuccinimide, mPEG propionaldehyde, and mPEG maleimide. In some cases, the branched PEG moiety or derivative comprises monomeric, dimeric and/or trimeric PEG moieties, or derivatives thereof. In some cases, the PEG moiety or derivative is trimeric methoxypolyethylene glycol maleimide.

In certain embodiments, the GLP-1r agonists or analogs thereof, have an increased half-life in vivo, e.g., at least 2 hours to 200 hours. For example, the PEGylated exenatide has a half-life in vivo of 2 hours, 6 hours, 12 hours, 20 hours, 24 hours, 36 hours, 48 hours, 60 hours, 72 hours, 88 hours, 100 hours, 112 hours, 124 hours, 150 hours, 175 hours, or 200 hours. In an exemplary embodiment, the compositions of the invention comprise a half-life in vivo of about 80-90 hours in non-human primates.

In certain embodiments the pharmaceutical composition comprising a therapeutically effective amount of at least one glucagon-like peptide 1 receptor (GLP-1r) agonist or analogs thereof, is administered systemically or locally. In certain embodiments, the administration of the pharmaceutical composition comprises subretinal injection, intravitreal injection, subconjunctiva injection, or intraocular injection.

Other aspects are described infra.

### Definitions

The terminology used herein is for the purpose of describing particular embodiments only and is not intended to be limiting of the invention.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the terms “effective amount” or “therapeutically effective amount” means a dosage sufficient to alleviate one or more symptoms of a disorder, disease, or condition being treated, e.g., Stargardt disease, dry and wet macular degeneration, also known as age-related macular degeneration (AMD or ARMD), juvenile macular degeneration, retinal degeneration, glaucoma, retinal dystrophy, branch and central retinal vein occlusion, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration including optic neuropathies such as anterior ischemic optic neuropathy, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behcet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alstrom syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome, or to otherwise provide a desired pharmacological and/or physiologic effect.

As used herein, a “long-acting GLP-1r agonist” is meant a GLP-1r agonist which is effective for at least one hour, at least six hours, at least twelve hours, at least one day, at least two days, at least one week, at least two weeks, at least one month, or at least two months.

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule.

As used in this specification and the appended claims, the term “or” is generally employed in its sense including “and/or” unless the content clearly dictates otherwise.

As used herein, the term “PEGylation” refers to a process of both covalent and non-covalent attachment or amalgamation of polyethylene glycol (PEG) polymer chains to molecules and macrostructures, such as a drug, therapeutic protein or vesicle.

As used herein, the term “pharmaceutically acceptable” refers to molecular entities and compositions that are “generally regarded as safe” e.g., that are physiologically tolerable and do not typically produce an allergic or similar untoward reaction, such as gastric upset and the like, when administered to a human.

As used herein, the terms “pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs disclosed herein, can also be incorporated into the described compositions and methods.

As used herein, the terms “subject” or “patient” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D are a series of images and graphs demonstrating that NLY01 treatment reduces avascular area and neovascularization area on postnatal day 17 (P17) oxygen-induced retinopathy (OIR) retina. FIG. 1A: Representative images of avascular area (red line) in OIR P17 retinal flatmount in vehicle- and NLY01-treated group. FIG. 1B: Representative images of neovascularization area in OIR P17 retinal flatmount. FIG. 1C: Quantitative results of avascular and neovascularization area (n=12). Data are presented as mean±SD and analyzed by unpaired t test. FIG. 1D: Quantitative results of avascular and neovascularization area (n=12). Data are presented as mean±SD and analyzed by paired t test. NLY01 treated mice exhibit reduction in pathologic neovascularization as well as avascular retinal area, indicating inhibition of vasodegeneration and promotion of reparative angiogenesis (revascularization of ischemic retina).

FIGS. 2A-2B are a series of graphs demonstrating that NLY01 reduces inflammatory and expression of genes known to promote pathologic angiogenesis in P15 OIR retina FIG. 2A: Inflammatory gene expression in vehicle- and NLY01-treated group (n=12). FIG. 2B: Angiogenesis-related gene expression in vehicle- and NLY01-treated group (n=12). Data are presented as mean±SD and analyzed by unpaired t test.

FIGS. 3A-3E are a series of images demonstrating the co-localization of NLY01 with microglia in OIR retina. FIG. 3A: Representative image of NLY01-FITC co-localization with microglia around the avascular area at P15. FIG. 3B: Representative image of NLY01-FITC co-localization with microglia around the neovascularization area at P15. FIG. 3C: 3D-reconstruction image of NLY01-FITC co-localization with microglia around the neovascular tufts. FIG. 3D: Representative image of NLY01-FITC co-localization with microglia around the avascular area at P17, demonstrating longer-term retention of NLY01 in the microglia. FIG. 3E: Representative image of NLY01-FITC co-localization with microglia around the neovascularization area at P17. Original magnification×200.

FIGS. 4A-4C are a series of plots and graphs demonstrating that NLY01 modulates microglia activation in P15 OIR retina. FIG. 4A: Representative FACS plots of CD11b⁺ CD45 low cells in P15 vehicle- and NLY01-treated retina. FIG. 4B: Quantification of CD11b⁺ CD45 low cells in P15 vehicle- and NLY01-treated retina (n=5). FIG. 4C: Inflammatory gene expression of ribosome RNA isolated from microglia/macrophages in vehicle and NLY01-treated Cx3cr1CreER:Rp122HA mice retina (n=7). Data are presented as mean±SD and analyzed by unpaired t test. NLY01-treated mice exhibited a reduction in microglia numbers and microglial proliferation, indicating modulation of microglia activation. In addition, microglia isolated from retinas of NLY01-treated mice exhibited reduced production of pathogenic inflammatory cytokines including TNF-α that are known to promote ocular inflammation.

FIGS. 5A-5E are a series of graphs and images demonstrating that NLY01 regulates inflammatory and hypoxic primary microglia activation including suppression of microglia expression of inflammatory genes and inhibition of activation of the pro-inflammatory transcription factor NFκB. LPS was used as a conventional inflammatory stimulator of microglia. FIG. 5A: Inflammatory gene expression of NLY01-treated primary brain microglia stimulated by LPS (n=3). FIG. 5B: Representative image of nuclear translocation after LPS treatment of primary brain microglia. Original magnification×400. FIG. 5C: Western blot analysis of NFkB p65 nuclear translocation in primary brain microglia after NLY01 treatment. NF-kB activation is known to occur via translocation from the cytosol to the nucleus. FIG. 5D: Pro-angiogenic gene expression of NLY01-treated primary brain microglia stimulated by hypoxia. FIG. 5E: Inflammatory and pro-angiogenic gene expression of NLY01-treated primary retinal microglia stimulated by LPS.

FIGS. 6A-6D are a series of graphs and images demonstrating that NLY01 does not show any angiogenic effect on human retinal endothelial cells (HRECs). FIG. 6A: Quantification of CTG Assay. FIG. 6B: Quantification of Caspases-3/7 assay. FIG. 6C: Representative image of PBS- and NLY01-treated HRECs tube formation. Original magnification×400. FIG. 6D: Quantitative results of tube formation (n=3). Data are presented as mean±SD and analyzed by unpaired t test.

FIG. 7 is a series of images and a graph demonstrating significant inhibition of laser-induced choroidal neovascularization (CNV) by intravitreal injection of NLY01 compared to PBS vehicle: a single-dose of 5 microgram of NLY01 was administered. N=16. Laser-induced CNV quantitated 7 days after intravitreal injection of NLY01.

FIG. 8 is a series of images and a graph demonstrating significant inhibition of laser-induced choroidal neovascularization (CNV) by subcutaneous injection of NLY01 compared to PBS vehicle. NLY01 (10 mg/kg) or vehicle was administered by subcutaneous injection immediately after the laser-injury and again 3 days later. Tissue was collected at 7 days after the laser-injury for quantitation of CNV. N=7 for PBS vehicle-treated mice, and N=8 for NLY01-treated mice.

FIG. 9 is a series of graphs demonstrating neuroprotection and rescue of retinal function by intravitreal injections of 1 microliter of NLY01 (5 microgram/microliter) vs. PBS vehicle at postnatal day 7 (P7), P14, P21, P28, and P35. N=8. ERG measurement performed at P30. NLY01 treatment improved both a-wave and b-wave, indicating neuroprotection of retinal neuronal elements and improvement of retinal function by NLY01.

FIG. 10 is a series of graphs demonstrating neuroprotection and rescue of retinal function by intraperitoneal injection of NLY01 (at either 10 mg/kg or 3 mg/kg) or PBS vehicle every 2 days. N=6. ERG measurement performed at P35. NLY01 treatment improved both a-wave and b-wave, indicating neuroprotection of retinal neuronal elements and improvement of retinal function by NLY01.

FIG. 11 is a study of retinal ganglion cell loss after retinal ischemia-reperfusion injury. The images show representative confocal images of NeuN-stained retina, displaying the number of surviving ganglion cells. The bar-graphs show quantitation of the NeuN-positive cells in mouse retinas. N=4 mice were used for each treatment condition. NLY01 treatment resulted in significant increase in retinal ganglion cell counts following retinal ischemia-reperfusion as compared to vehicle.

### DETAILED DESCRIPTION

The disclosure is based, in part, on the finding that glucagon-like peptide 1 receptor (GLP-1r) agonists are useful in the treatment of ocular diseases or disorders. In particular, long-acting GLP-1r agonists, such as for example, a PEGylated exenatidepeptide, NLY01. The PEGylated exenatide peptide is engineered to have a long circulation half-life while maintaining its biological potency for use in humans. In this disclosure, the high molecular weight PEGylated peptide is used for the treatment of ocular diseases including diabetic retinopathy, diabetic macular edema, branch and central retinal vein occlusion, dry and wet age-related macular degeneration, retinal degeneration, glaucoma, optic neuropathies, and other ocular conditions. Depending on the disease context, beneficial effects have been found to be both GLP-1r-dependent and GLP-1r-independent. In the mouse model of oxygen-induced retinopathy (OIR) model, a well-accepted model of ischemic retinopathies including diabetic retinopathy, NLY01 administration significantly reduced retinal ischemia (avascular retinal area) as well as pathologic pre-retinal neovascularization. This was associated with modulation of microglial activation and associated inflammatory changes. In the laser-induced choroidal neovascularization (CNV) model, a widely used model of wet age-related macular degeneration, NLY01 significantly reduced CNV area. In the rd10 model of retinal degeneration, NLY01 treatment significantly preserved neuroretinal function as assessed by electroretinography, indicating preservation of photoreceptor health as well as health of the inner retina. In the retinal ischemia-reperfusion model, NLY01 treatment significantly preserved retinal ganglion cell numbers, demonstrating a neuroprotective effect. All four of the above are clinical models of retinal diseases including chronic disease, in which long-acting drug provides a significant advantage which would revolutionize therapy for these and other chronic ocular conditions with greatly improved patient compliance—a treatment option that could utilize dosing of as long as once monthly treatment. Accordingly, certain embodiments include the development of a first in class, long-acting injectable peptide-based drug with disease-modifying effects in multiple ocular/retinal diseases.

Glucagon-Like Peptide 1 Receptor (GLP-1r) Agonists

Beyond its initial indication for glycemic control, Exenatide, an FDA-approved peptide (BYETTA™) and a glucagon-like peptide 1 receptor (GLP-1r) agonist, has been investigated for clinical indications in Alzheimer's disease (AD) and Parkinson's disease (PD) patients and animal models. Depending on the disease context, beneficial effects have been found to be both GLP-1r-dependent and GLP-1r-independent. In the clinical setting, a small number of PD patients, and results demonstrated improved motor and cognitive symptoms, indicating a potential novel PD therapy. However, exenatide has a short half-life (human −2 hr) and requires twice-daily subcutaneous (s.c.) injections that can be inconvenient and difficult for patients. A long-acting agent has been developed that offers similar pharmacological benefits to exenatide with significantly reduced dosing frequency, possibly even once monthly. NLY01 is a long-acting PEGylated exenatide with an extended half-life of 12 days in humans. The long-acting features of NLY01 are engineered through a unique half-life extension technology which still allows the composition to follow the same target and mechanism of action as exenatide (US20180369340, incorporated herein in its entirety). It is currently under clinical development for Type 2 Diabetes (T2D) and neurogenerative disorders including Parkinson's disease and Alzheimer's disease. Since NLY01 has a large molecular weight poly(ethylene glycol) polymer (PEG, 50,000 Da) conjugated to the small exenatide peptide (4,000 Da), similar pharmacological efficacy to exenatide in PD was not expected because of the likely inability to cross the blood-brain barrier (BBB). Strikingly, it was discovered that s.c. administered NLY01 demonstrates clear beneficial effects in PD animal models. Among its beneficial effects was the ability to modulate neuroinflammation in PD models, including modulation of reactive microglia that are believed to promote neurodegeneration.

A peptide agonist of GLP-1r, exenatide delays numerous neurodegenerative processes (Holscher, C. J Endocrinol, 2014. 221(1): p. T31-41) in addition to facilitating insulin release in T2D patients by stimulating GLP-1r. This peptide manages insulin release in a glucose-dependent manner and is therefore safe for non-diabetic patients. As a long-acting exenatide de-based therapy, NLY01 offers an improved drug delivery approach compared to exenatide, while maintaining its pharmacological effects. Because NLY01 has a large molecular weight poly(ethylene glycol) polymer (PEG, 50,000 Da) conjugated to the small exenatide peptide (−4,000 Da), similar pharmacological efficacy to exenatide in PD and AD was completely unexpected because of the likely inability to cross the blood-brain barrier (BBB) (Pardridge, W. M., NeuroRx, 2005. 2(1): p. 3-14). As described in detail below, it was unexpectedly discovered that subcutaneously-administered NLY01 accumulated significantly higher in the brain of PD and AD animal models and demonstrated clear beneficial effects in a number of newly established PD animal models (see also, Luk, K. C., et al., Science, 2012. 338(6109): p. 949-53) and AD animal models. The results demonstrated that the administration of NLY01 protected against alpha-synuclein preformed fibrils (PFFs)-induced loss of dopaminergic neurons, reduces the PFF-induced Lewy Body-like pathology, inhibits the PFF-induced reduction in striatal dopamine terminal density, and restored the behavioral deficits induced by PFFs as well as increases lifespan of Tg PD models. NLY01 significantly blocked microglia activation and decreased the formation of reactive astrocytes in the brain. Taken together, the findings clearly indicated that NLY01 has beneficial neuroprotective/disease-modifying effects against alpha-synuclein PFFs-induced behavioral deficits. Similarly, in Tg AD models, NLY01 treatment ameliorated memory impairment and reduced amyloid aggregation and tau formation, the hallmark of AD. Consistent with PD studies, NLY01 demonstrated significantly inhibited microglia activation and the population of reactive astrocytes in the AD brain.

Accordingly, in certain embodiments, the at least one GLP-1r agonist is a long-acting GLP-1r agonist or analogs thereof. In some embodiments, the at least one GLP-1r agonist comprises a polypeptide, an antibody, a nucleic acid, an aptamer, or a small molecule. In some embodiments, the at least one GLP-1r agonist or analogs thereof, comprise long-acting NLY01, liraglutide, exenatide, lixisenatide, albiglutide, dulaglutide, semaglutide, and taspoglutide. In some embodiments, the at least one GLP-1r agonist is NLY01.

In certain embodiments, the one or more agents embodied herein, e.g. NLY01 are therapeutically effective via GLP-1r-dependent and/or GLP-1r independent mechanisms. In some embodiments, the at least one agent comprises a polypeptide, an antibody, a nucleic acid, an aptamer, or a small molecule. In some embodiments, the at least one thereof, comprises long-acting NLY01, liraglutide, exenatide, lixisenatide, albiglutide, dulaglutide, semaglutide, and taspoglutide. In some embodiments, the at least one agent is NLY01.

In some embodiments, the GLP-1r agonist can be a polypeptide. For example, polypeptides include GLP-1 (7-36), GLP-1 (7-37) or GLP-1 (1-37), or variants thereof. See, for example, U.S. Pat. No. 8,758,761, which is incorporated by reference in its entirety. GLP-1 is rapidly, metabolized by a peptidase (dipeptidylpeptidase IV or DPP-IV). One way to counter the rapid degradation is to couple it to a fatty acid or PEG.

In addition, WO2007113205 and WO2006097538 also disclose a series of GLP-1 analogues or derivatives thereof produced by chemical modification or amino acid substitution, in which the most representative one is liraglutide. Liraglutide is a derivative of GLP-1, whose structure contains a. GLP-1 analogue of which the sequence is 97% homologous with human GLP-1, and this GLP-1 analog is linked with palmitic acid covalently to form liraglutide. The palmitic acid in the structure will form a certain steric hindrance to prevent the degradation by DPP-1V and to reduce renal clearance. The half-life of liraglutide in the human body administered by subcutaneous injection is about 10-14 hours. In some embodiments, it can be administered once on day and the daily dose is 0.6-1.8 mg.

Exenatide is a synthetic Exendin-4, with the trade name BYETTA™. Exenatide has been approved for the treatment of Type 2diabetes mellitus (T2DM) by the FDA. It has 50% homology with mammalian GLP-1 in sequence and has a similar affinity site of the receptor with GLP-1. (See Drucker D J, Nauck M A. Lancet. 2006, 368(9548):1696-1705). Exendin-4 has a longer half-life than GLP-1 and has recently been shown to have a hypoglycemic effect in humans when given twice a day for one month. Exenatide is a 39-amino acid peptide which closely resembles exendin-4. It is DPP-4 resistant and has many of the actions of GLP-1.

The incretin hormones GLP-1 and glucose-dependent insulinotropic peptide (GIP) are produced by the L and K endocrine cells of the intestine following ingestion of food. GLP-1 and GIP stimulate insulin secretion from the beta cells of the islets of Langerhans in the pancreas. Only GLP-1 causes insulin secretion in the diabetic state; however, GLP-1 itself is ineffective as a clinical treatment for diabetes as it has a very short half-life in vivo. Exenatide hears a 50% amino acid homology to GLP-1 and it has a longer half-life in vivo. Thus, it was tested for its ability to stimulate insulin secretion and lower blood glucose in mammals, and was found to be effective in the diabetic state. In studies on rodents, it has also been shown to increase the number of beta cells in the pancreas.

In some embodiments, the at least one long-acting GLP-1 agonist comprises an Fc-fusion GLP-1. (e.g. dulaglutide, efpeglenatide) or derivative thereof. In some cases, the long-acting GLP-1 agonist comprises an albumin-fusion GLP-1 (e.g. albiglutide) or derivative thereof. An example of an Fc-fusion GLP-1 composition used to treat PD or AD is dulaglutide. Dulaglutide is a glucagon-like peptide 1 receptor agonist (GLP-1 agonist) for the treatment of type 2 diabetes that can be used once weekly.

Dulaglutide consists of GLP-1(7-37) covalently linked to an Fc fragment of human Ig thereby protecting the GLP-1 moiety from inactivation by dipeptidyl peptidase 4. GLP-1 is a hormone that is involved in the normalization of level of glucose in blood (glycemia). GLP-1 is normally secreted by L cells of the gastrointestinal mucosa in response to a meal. Dulaglutide binds to glucagon-like peptide 1 receptors, slowing gastric emptying and increases insulin secretion by pancreatic Beta cells. Simultaneously the compound reduces the elevated glucagon secretion by inhibiting alpha cells of the pancreas, which is known to be inappropriate in the diabetic patient. An example of an albumin-fusion of GLP-1 composition used to treat PD or AD is albiglutide. Albiglutide is a glucagon-like peptide-1 agonist (GLP-1 agonist) drug used for treatment of type 2 diabetes. It is a dipeptidyl peptidase-4-resistant glucagon-like peptide-1 dimer fused to human albumin. Albiglutide has a half-life of four to seven days.

Additional GLP-1r agonists are disclosed in US2012048586 and include OAP-189; US20090098130; and US20160279199.

As used herein, in preferred aspects, a “GLP-1 receptor agonist” or a “GLP-1R agonist” or “GLP-1r agonist” is a molecule that increases the amount of activation of GLP-1R, producing effects similar to those produced by the naturally-occurring agonists GLP-1 and exendin-4. In certain aspects, GLP-1r agonists may increase the activation of GLP-1r, for example by binding to and activating GLP-1r, by causing a conformational change in the GLP-1R, by causing activation of a G protein coupled to the GLP-1R, by causing GLP-1R to remain in an activated (e.g., in the active conformation) condition for a longer period of time (including indefinitely), by mimicking the binding of naturally-occurring agonists, by modulating the binding of naturally-occurring agonists, by blocking inhibitors of GLP-1 or otherwise modulating GLP-1R activation or initiating the cascade of intracellular events that is characteristic of GLP-1R activation. Preferred GLP-1r agonists may increase activation of GLP-1r by at least 5%, at least 10%, at least 20%, at least 30%, at least 50%, at least 100%, at least 200%, or more, such as determined by an in vitro or other assay.

Suitable assays that may be employed to identify GLP-1r agonists are described for example in Thorkildsen, Chr. et al. (2003), Journal of Pharmacology and Experimental Therapeutics, 307, 490-496; Knudsen, L. B. et al. (2007), PNAS, 104, 937-942, No. 3; Chen, D. et al. (2007), PNAS, 104, 943-948, No. 3; and/or US2006/0003417 A1 (see Example 8). Briefly, in one suitable assay, a purified membrane fraction of eukaryotic cells harboring e.g. the human recombinant GLP-1 receptor, e.g. CHO, BHK or HEK293 cells, is incubated with the test compound or compounds in the presence of e.g. human GLP-1, e.g. GLP-1 (7-36) amide which is suitably labeled for example ¹²⁵I (e.g. 80 kBq/pmol). Varying concentrations of the test compound or compounds suitably may be used and the IC₅₀ values are determined as the concentrations diminishing the specific binding of human GLP-1, In a receptor functional assay, isolated plasma membranes from eukaryotic cells, as e.g. described above, expressing e.g. the human GLP-1 receptor are prepared and incubated with a test compound. The assay may be conducted by measuring cAMP as a response to stimulation by the test compound. In a reporter gene assay, eukaryotic cells such as described above, expressing e.g. the human CI LP-1 receptor and containing a multiple response element/cAMP response element-driven luciferase reporter plasmid are cultured in the presence of a test compound. cAMP response element-driven luciferase activities are measured as a response to stimulation by the test compound.

Suitable GLP-1r agonists include for example a bioactive GLP-1, a GLP-1 analog or a GLP-1 substitute, for example as disclosed in Drucker, D. J. (2006) Cell Metabolism, 3. 153-165; Thorkildsen et al., 003), Journal of Pharmacology and Experimental Therapeutics, 307, 190-496; Knudsen, L. B. et al. (2007), PNAS, 104, 937-942, No. 3; Chen, D. et al. (2007). INAS, 104, 943-948, No, 3; Liu, J. et al. (2007) Neurochem Int., 51, 361-369, No. 6-7; Christensen, M, et al, (2009), Drugs, 12. 503-513; Maida, A. et al, (2008) Endocrinology, 149, 5670-5678, No. 11 or US2006/0003417 A1. Exemplary compounds including for use in the present methods and compositions have been disclosed above and also may include GLP-1(7-37), GLP-1(7-36)amide, extendin-4, liraglutide, CJC-1131, albugon, allyiglutide, exenatide, exenatide-LAR, oxyntomodulin, lixisenatide, geniproside, AVE-0010, a peptide with GLP-1r agonistic activity or a small organic compound with GLP-1r agonistic activity.

Pharmaceutical Compositions

The compositions comprising at least one GLP-1r agonists or analogs thereof, can be prepared in a variety of ways known to one of ordinary skill in the art. Regardless of their original source or the manner in which they are obtained, the compositions can be formulated in accordance with their use. For example, the nucleic acids and vectors can be formulated within compositions for application to cells in tissue culture or for administration to a patient or subject. Any of the pharmaceutical compositions of the can be formulated for use in the preparation of a medicament, and particular uses are indicated below in the context of treatment.

These pharmaceutical compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This disclosure also includes pharmaceutical compositions which contain, as the active ingredient, polypeptides, nucleic acids and vectors described herein in combination with one or more pharmaceutically acceptable carriers. The term pharmaceutically acceptable carrier, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the disclosure, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

In some instances, the topical ocular formulation is a solution, a suspension, creams, ointments, gels, gel-forming liquid, suspension containing liposomes or micelles, spray formulation, or an emulsion. In some cases, the topical ocular formulation also includes one or more pharmaceutically acceptable excipients selected from stabilizers, surfactants, polymer base carriers, gelling agents, organic co-solvents, pH active components, osmotic active components and with or without preservatives. In some cases, the sustained release semi-solid formulation, sustained release solid formulation or ocular implant is injected into the affected eye. In some embodiments, the sustained release semi-solid formulation, sustained release solid formulation or ocular implant further comprises a pharmaceutically acceptable excipient. In some cases, the sustained release semi-solid formulation, sustained release solid formulation or ocular implant includes at least one or more GLP-1r agonists or analogs thereof, and a biodegradable polymer selected from polylactic acid (PLA), polyglycolic acid (PLGA) and polylactic acid and polyglycolic acid copolymers.

The pharmaceutical compositions can be formulated to further comprise at least one ophthalmically acceptable excipient such as, but not limited to, demulcent, tonicity adjusting agent, preservative, buffering agent, pH adjusting agent, solubilizing agent, surfactant, chelating agent, penetration enhancer, emulsifying agent, suspending agent, stabilizing agent, antioxidant, carrier, plasticizer, release modifying or controlling excipients, ion exchange resins and the like. Suitable demulcents include, but are not limited to, glycerin, polyvinyl pyrrolidone, polyethylene oxide, polyethylene glycol (PEG) such as but not limited to PEG 400, PEG 300 and the like or combinations thereof; propylene glycol, sorbitol and polyacrylic acid and the like or combinations thereof. Tonicity adjusting agents useful in the compositions of the present invention may include, but are not limited to, salts such as, but not limited to, sodium chloride, potassium chloride and calcium chloride, non-ionic tonicity agents may include, but are not limited to, propylene glycol, glycerol, mannitol, dextran and the like or combinations thereof.

Suitable chelating agents may include, but are not limited to, EDTA and its salts. Solubilizing agents, that may be employed include, but are not limited to, CREMOPHOR EL®, tween 80, cyclodextrin and the like or combinations thereof. Suitable cyclodextrins may be employed, such as, but not limited to, α-cyclodextrin, β-cyclodextrin γ-cyclodextrin, hydroxypropyl-β-cyclodextrin, hydroxypropyl-γ-cyclodextrin, dimethyl-β-cyclodextrin and dimethyl-γ-cyclodextrin, and the like or combinations thereof. pH adjusting agents may include sodium hydroxide, hydrochloric acid, boric acid, Tris, triethanolamine and sodium hydroxide. Suitable buffering agents include, but are not limited to, phosphates, acetates and the like, and amino alcohols such as 2-amino-2-methyl-1-propanol (AMP), ascorbates, borates, hydrogen carbonate/carbonates, citrates, gluconates, lactates, propionates and TRIS (tromethamine) buffers, and the like or combinations thereof. Suitable preservatives include, but are not limited to, benzalkonium chloride, polyquatemium-1, p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, sorbic acid, and the like or combinations thereof. Suitable penetration enhancers that may optionally be employed include, but are not limited to, polyoxyethylene glycol lauryl ether, polyoxyethylene glycol stearyl ether, polyoxyethylene glycol oleyl ether, sodium taurocholate, saponins, CREMOPHOR EL, and the like or combinations thereof.

Suitable surfactants that may be employed include, but are not limited to, ionic and nonionic surfactants, and the like or combinations thereof. Suitable nonionic surfactants include, but are not limited to, poloxamers, tyloxapol, polysorbates, polyoxyethylene castor oil derivatives, sorbitan esters, polyoxyl stearates and a mixture of two or more thereof. Suitable pharmaceutical carriers include sterile water; electrolytes such as sodium chloride; dextrose; dextrose in water or saline; lower alkanols, ointment bases such as but not limited to, natural wax e.g. white bees wax, camauba wax, wool wax (wool fat), purified lanolin, anhydrous lanolin; petroleum wax e.g. solid paraffin, microcrystalline wax; hydrocarbons e.g. liquid paraffin, white petrolatum (e.g. white PROTOPET®), yellow petrolatum, and the like or combinations thereof. Suitable emulsifying agent may be included such as, but not limited to, mono- or di-glyceride of a fatty acid, phosphatide, e.g., lecithin, polysorbates, macrogols, poloxamers, tyloxapol, polyethylene glycol derivatives, polyvinyl alcohol and the like, and mixtures thereof. Suitable stabilizing agent such as, but not limited to, polyethylene glycol hydroxystearate, thiourea, thiosorbitol, sodium dioctyl sulfosuccinate, monothioglycerol and the like, or combinations thereof may be employed. Antioxidants such as, but not limited to, ascorbic acid, acetylcysteine, cysteine, sodium hydrogen sulfite, butylated hydroxyanisole, butylated hydroxytoluene or alpha-tocopherol acetate may be employed. Plasticizers, such as, but not limited to, glycerol, and the like may be employed.

Release modifying or controlling excipients, such as but not limited to, polymeric release modifying or controlling excipients, non-polymeric release modifying or controlling excipients or combinations thereof may be included in the compositions of the present invention. Exemplary release modifying or controlling excipients include glyceryl behenate, chitosan, carrageenan, cellulose derivatives such as ethyl cellulose, acrylic acid and methacrylic acid polymers or copolymers and the like, or derivatives or combinations thereof.

The ophthalmic formulations may optionally include additional viscosity enhancing agents such as, but not limited to, cellulose and cellulose derivatives, such as, but not limited to, methylcellulose, hydroxypropyl cellulose, hydroxyethyl cellulose, ethylhydroxyethyl cellulose, hydroxypropylmethyl cellulose, sodium carboxymethyl cellulose, cellulose acetophthalate, and the like or combinations thereof; alginic acid, sodium alginate, propylene glycol alginate, polyvinylpyrrolidone, carboxyvinyl polymers or carbomers (CARBOPOL®), polyvinyl alcohol, glycerin, polyethylene glycol, triblock copolymers of polyoxypropylene and polyoxyethylene, polyethoxylated sorbitan, polysorbate 80, chondroitin sulfate, dimethicone, perfluorononyl dimethicone, cyclomethicone, dextrans, proteoglycans, natural polysaccharides, such as, but not limited to, hyaluronic acid and salts thereof, guar gum, karaya, xyloglucan gum, chitosan, gellan gum, pectin, collagen, modified collagen and like or combinations thereof.

The ophthalmic formulations o may optionally include additional gelling agents such as, but not limited to, polysaccharide gums such as, but not limited to, gellan gum, tamarind gum, tragacanth, locust bean gum, agarose, carageenans, guar gum, hydroxypropyl guar gum, hyaluronic acid, chitosan, konjac, acacia, pectin, arabic, curdlan, glucan gum, scleroglucan and sulfated glucan sulfate and the like or combinations thereof; cellulose and its derivatives such as, but not limited to, methyl cellulose, carboxymethyl cellulose, methyl cellulose, hydroxypropyl cellulose, methyl hydroxypropyl cellulose, hydroxypropyl methyl cellulose, cellulose acetate, ethyl cellulose, methyl hydroxyethyl cellulose, hydroxyethyl cellulose, cellulose gum, and the like or combinations thereof; cross-linked acrylic polymers or carbomer (CARBOPOL™), aloe vera gel, polyvinyl alcohol, polyacrylamide, poloxamer, polymethylvinylether-maleic anhydride, swellable water-insoluble polymers such as, but not limited to, hydrogel and the like or combinations thereof. Ion exchange resins such as, but not limited to, inorganic zeolites or synthetically produced organic resins may be employed in the compositions of the present invention. The ophthalmic formulations of the present invention may optionally include additional mucodhesive agents such as, but not limited to, polyacrylic acid, hyaluronans, chitosan, pullulan, cellulose derivatives such as, but not limited to, methyl cellulose, hydroxypropyl methyl cellulose, sodium carboxymethylcellulose, poly (galacturonic) acid, sodium alginate, pectin, xyloglucan, xanthan gum, carbomers (CARBOPOL™), polyvinyl alcohol, polyvinylpyrrolidone, polyethylene glycol, poloxamer, and the like or combinations thereof.

The above listing of examples is given for illustrative purposes and is not intended to be exhaustive. Examples of other agents useful for the foregoing purposes are well known in ophthalmic formulation and are contemplated by the present invention. It is also contemplated that the concentrations of the excipients in the formulations of the present invention can vary. The ophthalmic formulations of the present invention can be in the form of eye drops, eye lotions, suspensions, dispersions, gels, ointments, emulsions, colloidal solutions, ocular inserts, ocular hydrogels, films, minitablets, nanoemulsions, and particulate systems such as but not limited to, liposomes, microparticles, nanoparticles, and the like. In one embodiment, the ophthalmic formulation of the present invention is in the form of an in-situ gelling system. In another embodiment, the in-situ type gelling composition of the present invention may comprise one or more cross-linking agent, such as but not limited to borate, and the like. In another embodiment, the in-situ type gelling composition of the present invention does not comprise one or more cross-linking agent.

In a further embodiment, the ophthalmic formulations are provided in the form of an ocular insert, such as a bioerodible ocular insert. In another embodiment, the ophthalmic formulations are in the form of ocular insert is a non-bioerodible ocular insert.

The ophthalmic formulations of the present invention may be in the form of liquid, solid or semisolid dosage form. Further, in one embodiment, the ophthalmic formulations of the present invention are formulated so as to have a pH and osmolality that are compatible with the eye. The ophthalmic formulations of the present invention may comprise depending on the final dosage form suitable ophthalmically acceptable excipients. In one embodiment, the ophthalmic formulations are formulated to maintain a physiologically tolerable pH range. In one embodiment, the pH range of the ophthalmic formulation is in the range of from 5 to 9. In another embodiment, pH range of the ophthalmic formulation is in the range of from 6 to 8.

In a further embodiment, the ophthalmic formulations are for topical administration to the eye. In another embodiment, the ophthalmic formulations are for intraocular or periocular administration. In a further embodiment, the ophthalmic formulations are for immediate release of active agent in the ocular cavity.

In another embodiment, the ophthalmic formulations are for sustained or controlled release in the ocular cavity. In a further embodiment, the ophthalmic formulations are for at once-α-day administration. In certain embodiments, the ophthalmic formulations are once every two days, or once every three days, or once every four days, or once every five days, or once every six days, or once weekly, or once every eight days, or once every nine days, or once every ten days, or once every eleven days, or once every twelve days, or once every thirteen days, or once every fourteen days and the like. In certain embodiments, the ophthalmic formulations are once every month, or once every six weeks or once every two months. In one embodiment, the sustained or controlled release delivery of the active agent from the ophthalmic formulation is for a sustained period of time of about 24 hours. In another embodiment, the sustained or controlled release delivery of the active agent from the ophthalmic formulation is for a sustained period of time of about 12 hours. In a further embodiment, the sustained or controlled release delivery of the active agent from the ophthalmic formulation is for a sustained period of time of about 10 hours. In yet another embodiment, the sustained or controlled release delivery of the active agent from the ophthalmic formulation is for a sustained period of time of about 8 hours. In one embodiment, the sustained or controlled release delivery of the active agent from the ophthalmic formulation is for a sustained period of time of about 6 hours. In a further embodiment, the sustained or controlled release delivery of the active agent from the ophthalmic formulation is for a sustained period of time of about 4 hours to about 24 hours.

Depending on the dosage form of the ophthalmic formulations of the present invention, appropriate method of preparation is employed. Various methods for preparation of ophthalmic formulations known in the art may be employed. Further depending on the dosage form, the ophthalmic formulations or excipients and/or active agents employed therein are suitably sterilized by one or more methods known to a person skilled in the art. In one embodiment, the ophthalmic formulations in the form of ocular insert, is prepared by molding or extrusion procedures well known in the art. In another embodiment, the ophthalmic formulation in the form of ophthalmic solution is prepared by either by dissolving or suspending prescribed amount of a drug in a prescribed volume of a carrier solvent along ophthalmically acceptable excipients. Particle size of certain ophthalmic formulations of the present invention is within ophthalmically acceptable limits known to a person skilled in the art.

The compositions of the present invention are useful for the treatment of humans or animals.

The active substance(s) of the instant disclosure, e.g. at least one or more GLP-1r agonists or analogs thereof, may be administered orally, topically, and parenterally, in dosage unit formulations containing conventional non-toxic pharmaceutically acceptable carriers. For oral administration in the form of a tablet or capsule, the active substance may be combined with nontoxic, excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

For topical administration, active substance(s) can be incorporated into various types of ophthalmic formulations for topical delivery to the eye. They may be combined with ophthalmologically acceptable preservatives, surfactants, viscosity enhancers, penetration enhancers, buffers, sodium chloride, and water to form aqueous, sterile ophthalmic suspensions or solutions. Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solutions may contain a thickener, such as, hydroxymethylcellulose, hydroxyethylcellulose, hydroxypropylmethylcellulose, methylcellulose, polyvinyl-pyrrolidone, or the like, to improve the retention of the formulation on the eye and thereby facilitate absorption into the inside of the eye.

Embodiments of the invention may be delivered parenterally, i.e., by intraocular, intravitreal, or subcutaneous (s.c.), by direct injection. In some embodiments, it will be understood by one of ordinary skill in the art that generally, dosage ranges may be determined based on the method of application of the GLP-1r agonists or analogs thereof. In one aspect, suitable dosage ranges include 1-1000 milligrams daily, alternately 10-500 milligrams daily, and optionally 50-500 milligrams daily, depending as usual upon the exact mode of administration, form in which administered, the indication toward which the administration is directed, the subject involved and the body weight of the subject involved, and the preference and experience of the physician in charge.

The formulations comprising the one or GLP-1r agonists or analogs thereof, may be delivered in a number of drug delivery vehicles. In some embodiments, the formulations comprising the one or GLP-1r agonists or analogs thereof may be delivered to the ocular region through a drug delivery vehicle, such as an intravitreal insert, or intraocular delivery system which may or may not be bioerodible. The intravitreal insert delivers sustained sub-microgram levels of the formulations comprising the one or GLP-1r agonists or analogs thereof, to the eye. The intravitreal insert may have a relatively high dosage or a relatively low dosage. The intravitreal insert can be configured to deliver a therapeutic effect for variable durations. For example, the dosage duration may be at least 3 months, 6 months, 12 months, at least 18 months, at least 24 months, at least 30 months and/or at least 36 months.

The intravitreal insert can provide sustained delivery of sub-microgram levels of formulations comprising the one or GLP-1r agonists or analogs thereof, over time. For example, in some embodiments, the intravitreal insert may deliver the formulations comprising the one or GLP-1r agonists or analogs thereof, for a dosage duration of up to at least 12 months, at least 24 months, and/or at least 36 months. In other exemplary embodiments, the low-dose intravitreal insert may deliver at least 0.5 μg per dose, at least 0.75 μg per dose, at least 1 μg per dose, or at least 2 μg per dose for a dosage duration of at least 3 months, 6 months, at least 12 months, at least 24 months, and/or at least 36 months.

The intravitreal insert may be inserted into the eye using a conventional ocular insertion device. For example, the intravitreal insert may be inserted using a device with a 25, 27 or 30-gauge needle. Typically, the insertion procedure is non-surgical and may be performed in a retinal specialist's office. For insertion, the needle of the device is inserted into the eye through the pars plana. The needle is inserted to about the equator of the eye and then the plunger of the device is depressed such that the intravitreal insert or intraocular delivery system is inserted into the vitreous of the eye. After injection, the insert or delivery system settles inferiorly at the posterior portion of the eye (distal the pars plana) at or near the vitreous base of the eye or generally in a region of the posterior segment that is outside of the visual axis. In different embodiments, the intravitreal insert may be positioned in alternative locations and be of different forms including an amorphous mass which may or may not bioerode.

In certain embodiments, the formulations comprising the one or GLP-1r agonists or analogs thereof, are used in treating or inhibiting degradation of the retina of a subject in need. One such disease that effects the retina is diabetic retinopathy. Diabetic retinopathy is a microvascular complication of diabetes mellitus. Diabetic retinopathy is a disease in which damage occurs to the retina due to high blood sugar levels. Blood vessels may swell, leak, or close, which stops blood from passing through. Leaking of the blood vessels may cause swelling of the retina including the macula. Swelling of the macula is defined as diabetic macular edema. Diabetic retinopathy may also lead to the deterioration of other parts of the retina including the retinal pigment epithelium, or RPE. RPE lies between the choroid and neurosensory retina to form the blood-retinal barrier. RPE serves an important barrier to maintain retinal health and include a collection of interrelated structures and activities that regulate the transepithelial movement of solutes, including: diffusion through the paracellular spaces, facilitated diffusion through the cells, active transport, receptor-mediated and bulk phase transcytosis, and metabolic processing of solutes in transit. RPE function is essential to maintaining a dehydrated neural retinal.

In certain embodiments, the formulations comprising the one or GLP-1r agonists or analogs thereof, are used in treating and inhibiting degeneration of the retina of a subject in need where the patient is suffering from retinitis pigmentosa and/or choroidal neovascularization. Retinitis pigmentosa is a group of diseases in which one of many different mutations causes rod photoreceptor cell death resulting in night blindness. With so many different pathogenic mutations, it appears that there are many different mechanisms by which rod cell death occurs. Regardless of the mutation and the mechanism of rod cell death, after rods die, cone photoreceptors undergo gradual degeneration resulting in gradual constriction of visual fields and eventual blindness.

The intravitreal insert may comprise biocompatible polymers or copolymers which may or may not also be bioerodible including, but not limited to, Dexon, Vicryl, Polysorb, poly(lactide-coglycolide) (PLGA), polyglycolic acid (PGA), polylactic acid (PLA), polycaprolactone (PCL), polydioxanone (PDO), polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, polyvinyl acetates, plasticized ethylene vinylacetate copolymer, polyvinyl alcohol, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4-isopropylidene diphenylene carbonate), vinylidene chloride, acrylonitrile copolymer, vinyl-chloride-diethyl fumerate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer, vinylidene chloride-acrylonitrile copolymer, etc. In preferred embodiments, the intravitreal insert comprises such as poly(lactic-co-glycolic acid). In alternative embodiments, the intravitreal insert may form from a non polymeric matrix.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### EXAMPLES

### Example 1: A Long-Acting GLP1R Agonist Ameliorates Retinal Angiogenesis in the Oxygen-Induced Retinopathy Model

GLP1R agonists have been found to be vasculoprotective and anti-inflammatory in multiple disease models, such as cardiac and retinal ischemia reperfusion, but its role in retinal angiogenesis remains unclear. This study investigated whether NLY01, a long acting glucagon-like peptide 1 receptor (GLP-1R) agonist could promote re-vascularization of the retinal vasculature and ameliorate pathologic retinal neovascularization in the oxygen-induced retinopathy (OIR) model, the most widely used model for studying ischemic retinal conditions including diabetic retinopathy.

### Materials and Methods

Animals

C57BL/6J mice, CX3CR1^{YFP-CreER/YFP-CreER} (Cat. No. 021160) mice and RiboTag⁴ (B6J.129(Cg)-Rpl22tm1.1Psam/SjJ, #029977) mice were purchased from the Jackson laboratory. The animals were maintained on an AIN-76A diet and water ad libitum and housed at a temperature range of 20-23° C. under 12:12-h light-dark cycles. All the animal procedures were approved by the Institutional Animal Care and Use Committee of the Johns Hopkins University School of Medicine and were conducted in accordance with the Association for Research in Vision and Ophthalmology Statement for the Use of Animals in Ophthalmic and Visual Research.

Mouse Model of Oxygen-Induced Retinopathy and NLY01 Treatment

The mouse OIR model was described previously. Pups were placed in 75% oxygen chamber with their nursery mother for 5 days from postnatal day 7 (P7) to P12. At P12, mice were removed from the chamber and returned to room air. For the NLY01 treatment, pups received intravitreal injection of NLY01 (5 μg/μl) or NLY01-FITC in one eye, and PBS in the contralateral eye as control. Mice were euthanized at different time points for further experiments.

Retinal Whole-Mount Preparations and Staining

Retinal whole-mount staining was performed as previously described. Briefly, mice were euthanized at P17. Mice with body weight lower than 6 g (P17) were excluded from analysis. Whole eyes were fixed in 4% paraformaldehyde for 30 minutes and the retinas were carefully dissected. After blocking with 10% normal goat serum in PBST (PBS plus 3% Triton X-100), the retinal vessels were visualized by incubating overnight with Alexa Flour 594-conjugated isolectin GS-IB4 from Griffonia simplicifolia (GS lectin) (Life Technologies). For the microglia and endothelial cell staining, retinas were incubated with rabbit anti-mouse lba1 antibody and rat anti-mouse CD31 antibody 4° C. overnight. The retinas were then extensively washed in PBST for 3 h with changes of PBST every 30 min, followed by labeling with goat anti-rabbit IgG conjugated with Alexa Fluor 647 antibody (Invitrogen, Carlsbad, Calif.) and goat anti-rat IgG conjugated with Alexa Flour 594 at 4° C. overnight. After washing with PBST, retinas were flat-mounted on slides in Fluoromount-G (Electron Microscopy Sciences, Hat-field, PA). Photographs were taken using a Zeiss LSM (LSM 710, Carl Zeiss, Thornwood, N.Y., USA) with 20× objective lens. Imaris software (version 8.1.2: Bitplane AG) was used for 3D reconstructions of the confocal Z-stacks.

Quantitation of Retinal VO and Pathologic NV

Retinal VO and NV were quantitated as previously described. Briefly, images of each of the four quadrants of retina were imported into Adobe Photoshop (Adobe System Inc., San Jose, Calif.). Retinal segments were merged to produce one image of each entire retina. VO and pathologic NV areas were quantified by comparing the number of pixels in the VO or NV area with total number of pixels in the whole retina area.

Real-Time PCR Analysis

RNA was isolated using RNeasy mini kit (Qiagen, Valencia, Calif., USA) and single-stranded cDNA was synthesized using MMLV Reverse Transcriptase (Invitrogen). qPCR was performed with SYBR Green PCR Master Mix (Applied Biosystens) with StepOnePlus real-time PCR system (Applied Biosystems). The qPCR primers were TNF-α; IL-6; IL-10; Cxcl2; IL-18; Vegf; Ang2. ppia: was used for normalization.

Flow-Cytometry for Whole Retina

Retinas were collected at P15 or P17 and then digested with papain (1.65 U/ml, Worthington Biochemical, Lakewood, N.J.) by incubating in 37° C. water bath for 16 minutes with gentle swirl of tube every 8 minutes. Tissues were then disaggregated by gentle pipetting up and down, and dropwise passing back into the tube through a serological pipette in low-ovomucoid (Lo-Ovo) solution. Sing cell suspensions of tissues were obtained by passing through 70 mm cell strainer (BD Biosciences, San Diego, Calif.). The cells were then washed twice with PBS containing 1% FBS, 10% NaN3, and HEPES (10 mM) and blocked with rat anti-mouse CD16/32 antibody (BD Biosciences, San Diego, Calif.) for 5 minutes in 4° C. Cells were incubated with rat anti-mouse APC-CD11b (Clone M1/70, eBioscience, San Diego, Calif.) and FITC-CD45 (eBioscience, San Diego, Calif.) for 30 minutes in room temperature. Cells were washed for three times. A four-laser Sony SH800 cell sorter (Sony Biotechnology, San Jose, Calif.) was used to collect the data, and FlowJo software (FlowJo, LLC., Ashland, Oreg.) was used for analysis.

Ribosome Immunoprecipitation (IP)

Isolation of polysome-bound mRNA using RiboTag from tissues was performed as previously described. Retinas were homogenized in 400 μL polysome buffer (50 mM Tris, pH 7.5, 100 mM KCl, 12 mM MgCl₂, 1% NP40, 1 mM DTT, 200 U/mL RNasin, 1 mg/mL Heparin, 100 μg/mL cyclohexamide and 1× protease inhibitor) using pellet pestles (Kimble Chase) on ice. The homogenate was centrifuged at 12000 rpm for 10 min at 4° C., the supernatant were collected for input and immunoprecipitation with HA antibody. Briefly, 25 μL of anti-HA antibody-conjugated magnetic beads (M180-11, MBL) were added to the retinal supernatant, and incubated at 4° C. overnight on an orbital shaker. The next day, the beads were washed 4 times with high salt buffer (50 mM Tris, pH7.5, 300 mM KCl, 12 mM MgCl₂, 1% NP40, 1 mM DTT, 100 μg/mL cyclohexamide). Beads were then resuspended in 350 μL RLT plus beta-mercapotoethanol, thoroughly vortexed and pelleted using a magnetic separator. The supernatant was transferred into a new tube and RNA was extracted using RNeasy Micro kit (Qiagen), according to manufacturer's instructions.

Primary Brain Microglia Isolation

Primary microglia were isolated from brain as previously reported. Briefly, brain tissues from C57 mice at P1 were obtained and washed with complete medium (DMEM/F12 supplemented with 10% heat-inactivated FBS, 50 U ml-1 penicillin, 50 ug ml-1 streptomycin, 2 mM L-Glutamine, 100 μM non-essential amino acids with 2 mM sodium pyruvate) for three times. Brain tissues were then digested with 0.25% trypsin-EDTA in 37° C. for 10 minutes. After being washed for 3 times with complete medium, single-cell suspension was obtained by trituration. Cell debris and aggregates were removed by passing the single-cell suspension through a 70-μm nylon mesh. The final single-cell suspension thus achieved was cultured in T75 flasks for 13 days, with a complete medium change on day 6. Microglia were isolated using the EasySep Mouse CD11b Positive Selection Kit (StemCell).

Primary Retinal Microglia Isolation from Immortomouse

Immortalized mouse retinal microglia were isolated as described (S Dharmarajan et al., J Neuroinflammation 2017; 14:76). Cells at passages 11-14 were grown in DMEM/F-12 (Thermo Scientific, Waltham, US) medium supplemented with 10% FBS (Invitrogen, Carlsbad, Calif.), 1% Penicillin/Streptomycin (Sigma, St Louis, Mo.) and 1% MEM NEAA (Gibco, Invitrogen Corp, USA). Cells were maintained in a humidified 5% CO₂ incubator at 31° C. throughout the entire study. Media were changed every 2-3 days.

Cell Culture and Treatment

For LPS experiments both primary brain and retinal microglia were cultured with incomplete medium (DMEM/F12 supplemented with 50 U ml⁻¹ penicillin, 50 μg ml⁻¹ streptomycin, 2 mM L-Glutamine, 100 μM non-essential amino acids with 2 mM sodium pyruvate) overnight. Cells were pretreated with NLY01 (1 μM) for one hour followed by LPS treatment at a concentration of 100 ng/mL for the times specified in the respective figure legends. Human retinal endothelial cells (HRECs; Cell Systems, Kirkland, Wash.) were cultured in EGM2-MV medium (Lonza) in a humidified 5% CO₂ incubator at 37° C., and medium was changed every 2-3 days. HRECs were grown in fibronectin (Invitrogen, Carlsbad, Calif.) coated dishes and were used at passages 6-10. For NLY01 treatment, cells were cultured in EGM2 without fetal bovine serum (FBS) (Invitrogen) overnight and then treated with NLY01 (1 μM) overnight.

For hypoxia experiments, primary brain microglia were exposed to either normoxia or 1% oxygen (hypoxia) for 24 hours. NLY01 was added immediately before the exposure to hypoxia. Experiments were done in triplicate.

Immunofluorescence Staining of NF-κB in Primary Microglia

Cells were seeded in 35 mm dish at the density of 10⁵ per well. After treatment, cells were fixed with 4% paraformaldehyde for 20 minutes at room temperature. After washing with PBS, cells were permeabilized with 0.5% Triton X-100 in PBS for 5 minutes. Cells were then blocked with 4% BSA in TBST (TBS with 0.05% Tween) for 60 minutes in room temperature. Cells were incubated with rabbit anti-mouse NF-kB p65 (Cell Signaling) and goat anti-mouse lba1 (Invitrogen) for 60 minutes at room temperature. After being washed three time with TBST, cells were then incubated with donkey anti-rabbit IgG conjugated with Alexa Flour 594 and donkey anti-goat IgG conjugated with Alexa Flour 647. Cells were washed 3 times with TBST and followed by DAPI staining. Photographs were taken using a Zeiss LSM (LSM 710, Carl Zeiss, Thornwood, N.Y., USA) with 40× objective lens.

Nuclear Extract Preparation and Western Blot Analysis

Nuclear extracts from primary microglia were prepared using NE-PER Nuclear and Cytoplasmic Extraction Reagents (ThermoFisher Scientific) following the manufacturers' instructions. Proteins were quantified by the BCA protein assay (BioRad) and protein extracts were aliquoted and stored at −80° C. Rabbit anti-mouse NF-κB p65 antibody (Cell Signaling) was used at 1:1000 dilution. Lamin B antibody (1:400) (Santa Cruz Biotechnology) was used for loading control normalization. After incubating with primary and secondary antibodies, the blots were detected with Femto Chemiluminescent Substrates (Thermo Scientific, Waltham, Mass.).

Cell Proliferation Assay and Apoptosis Assay

HREC proliferation activity was measured using the CellTiter 96 AQueous One Solution Cell proliferation assay (Promega Corp) following the manufacturer's instruction. For the measurement of in vitro apoptosis, caspase-3/7 activity was measured using a Caspase-Glo 3/7 assay kit (Promega Corp., Madison, Wis.) according to the manufacturer's instructions. After incubation, the plates were shaken thoroughly, and absorbance was measured using a microplate reader (FLUO-star OPTIMA; BMG Labtech GmbH, Ortenberg, Germany) at 492 and 620 nm.

Tube Formation Assay

HREC tube formation was evaluated using a two-layer collagen gel mixture assay, as previously described. HRECs were trypsinized, and 7×10⁴ cells were seeded on top of the lower collagen gel layer in each well of the 24-well plate. After overnight culture, cells were then treated with NLY01 in EBM2 plus2% fetal bovine serum for 5 hours. Medium was then removed, and 150 μL of collagen gel mixture was added to each well. After 2 hours of gel polymerization at 37° C., 500 μL of medium containing NLY01 was added to the upper layer of collagen gel. Eighteen hours later, six random fields in each well were chosen and photographed using an Axiovert 200M microscope (Carl Zeiss Microscopy LLC).

Statistical Analysis

Data were presented as mean±SD. Student's t-test was used to perform statistical analysis. Multiple groups were compared by using one-way analysis of variance. P values less than 0.05 was considered statistically significant.

Results

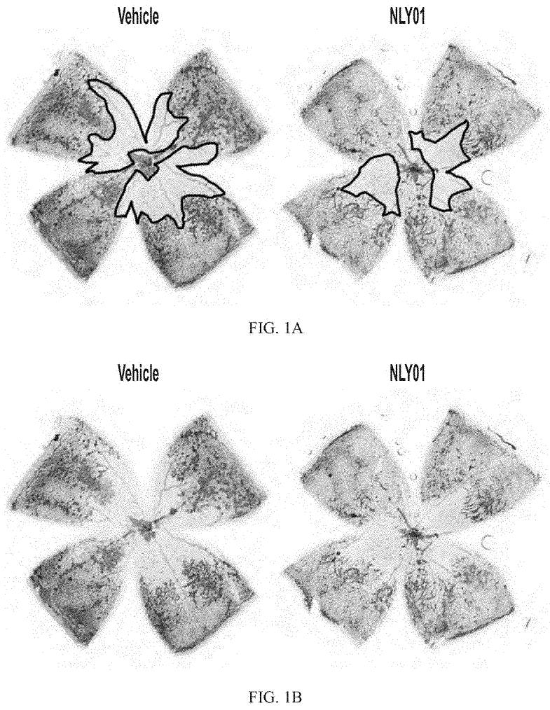

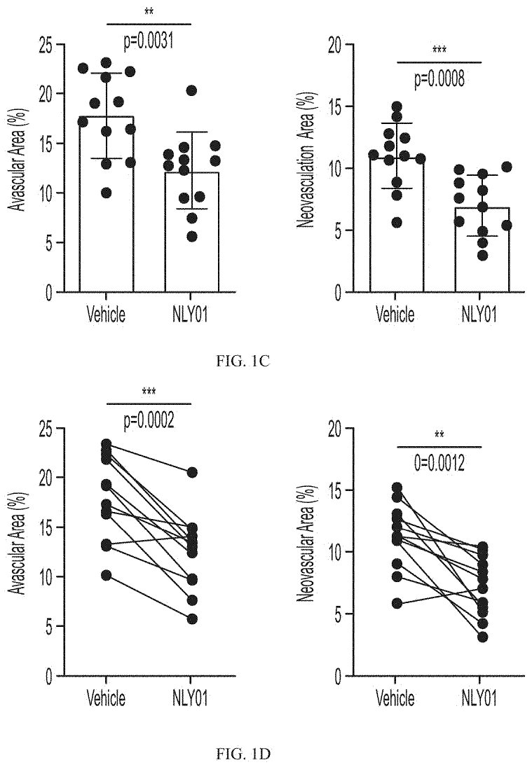

NLY01 ameliorates vascular degeneration, improves vascular recovery, and reduces pathological neovascularization in oxygen-induced retinopathy model. It was examined whether NLY01 has any protective effect in the oxygen-induced retinopathy, one of the most frequently used models for ischemic retinal diseases including proliferative diabetic retinopathy. In this model, hyperoxic exposure leads to vaso-obliteration in central retina. After returning to room air, retinal ischemia stimulates multiple factors and leads to pathological neovascularization. The pathology reaches the maximum severity at P17 (1). Comparing the vehicle-treated eye with NLY01-treated eyes, the results showed reduced avascular (ischemic) retinal area (FIGS. 1A,1C). Ameliorated pathological neovascularization was observed in the NLY01-treated eyes (FIGS. 1B, 1C). The avascular area and neovascularization area between NLY01-treated eyes and their corresponding contralateral vehicle-treated eyes was compared. Significantly reduced avascular area and neovascularization area in NLY01-treated eyes compared to vehicle-treated eyes (FIG. 1D) was observed for virtually all mice providing evidence that NLY01 ameliorates vascular degeneration, improved reparative angiogenesis, and inhibits pathological neovascularization in oxygen-induced retinopathy model of ischemic retinopathy.

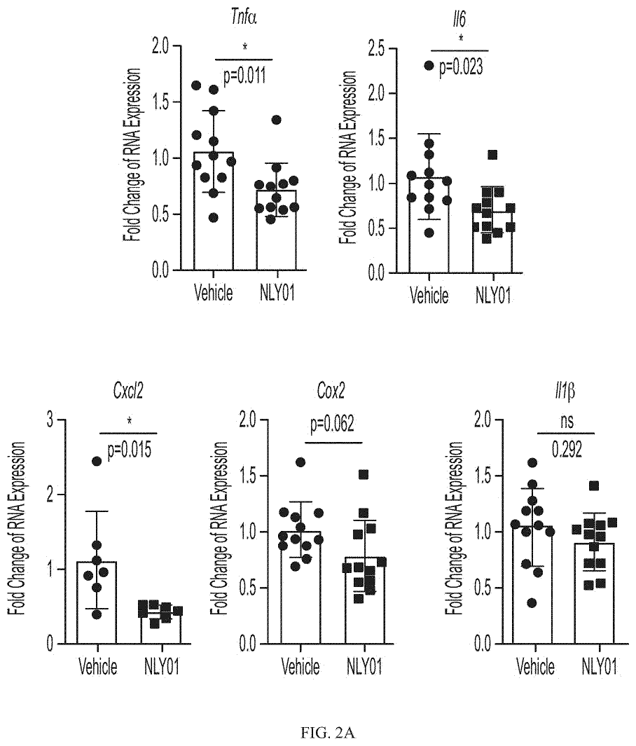

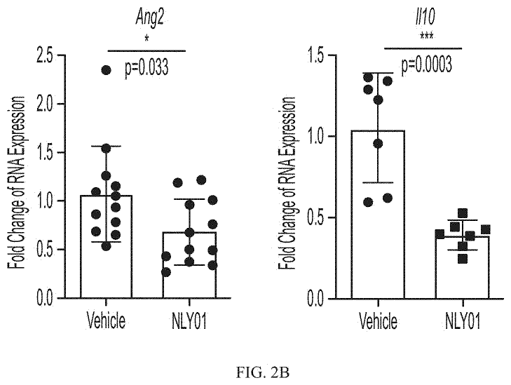

NLY01 treatment reduces inflammatory and vasculopathic gene expression in retina. Retinas were collected at P15 to evaluate the inflammatory cytokines expression after NLY01 treatment. The expression of Tnfα, IL-6, COX2, and Cxcl2 were reduced compared to the vehicle-injected eyes (FIG. 2A). Tnfα and IL-6 are classical inflammatory cytokines closely involved in the pathophysiology and progression of DR (2). Cxcl2 found to be increased in the vitreous of PDR patient (3), plays important role in recruiting inflammatory cell and amplifying inflammation. It was also investigated whether other genes related to vascular function (FIG. 2B). It was found that NLY01 reduced the expression of angiopoietin 2 (Ang2), a factor known to regulate pathologic ocular angiogenesis and vascular leakage. The suppression of IL-10 was also observed in NLY01-treated eyes. IL-10 has been found to be pro-angiogenic in multiple ocular pathologies.

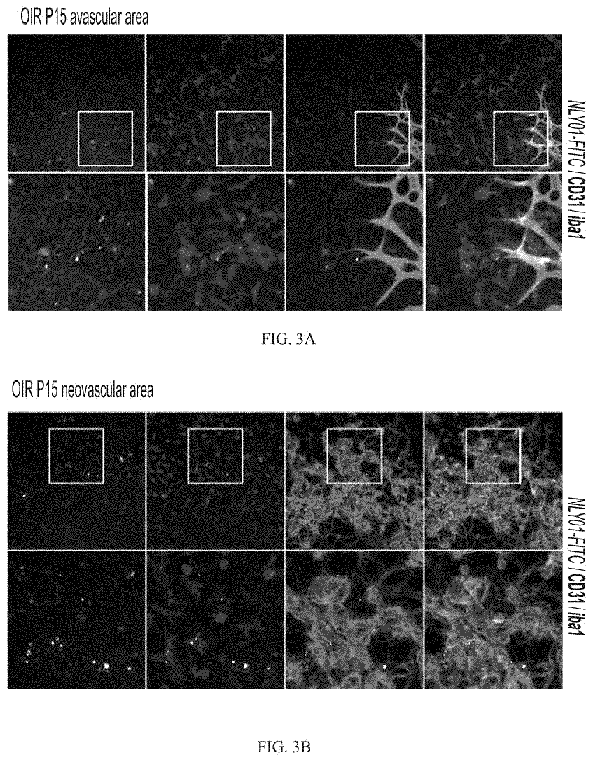

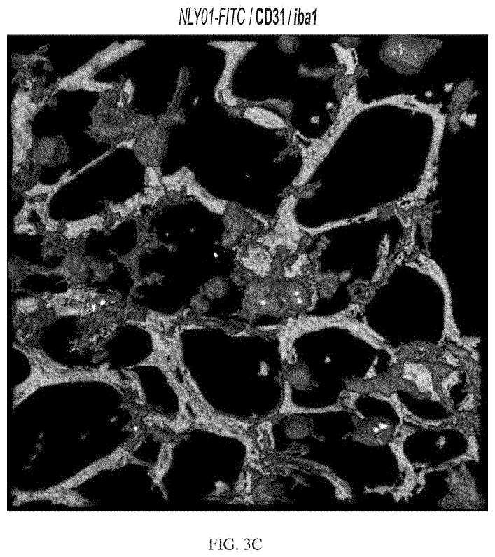

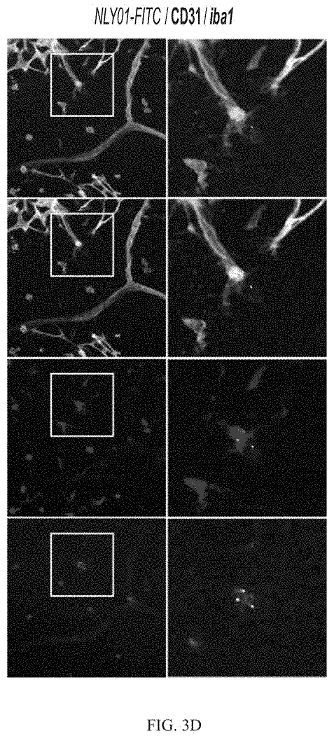

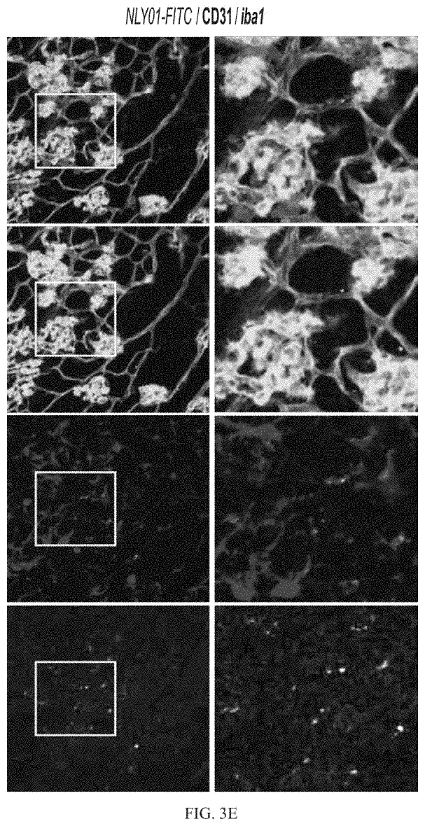

NLY01 co-localizes with microglia. To further determine the potential mechanism involved, the distribution of NLY01 was examined in retina by injecting FITC-tagged NLY01 at P12 and visualizing its localization at P15 and P17. At P15, it was found that the FITC-NLY01 distributed around both the avascular and neovascularization area. FITC-NLY01 were mainly co-localized with lba1⁺ microglia/macrophages (FIGS. 3A, 3B). With Z-stack and Imaris 3D reconstruction, the co-localization of FITC-NLY01 with lba1⁺ cells was confirmed (FIG. 3C), providing evidence that the FITC-NLY01 were mainly co-localized by the retinal microglia/macrophages. After 5 days of injection at P17, FITC-NLY01 were still maintained in the retina, and showed the similar pattern of co-localization with the lba1⁺ cells in the avascular and neovascular area as P15, demonstrating long-term retention of NLY01 in microglia. (FIGS. 3D, 3E)

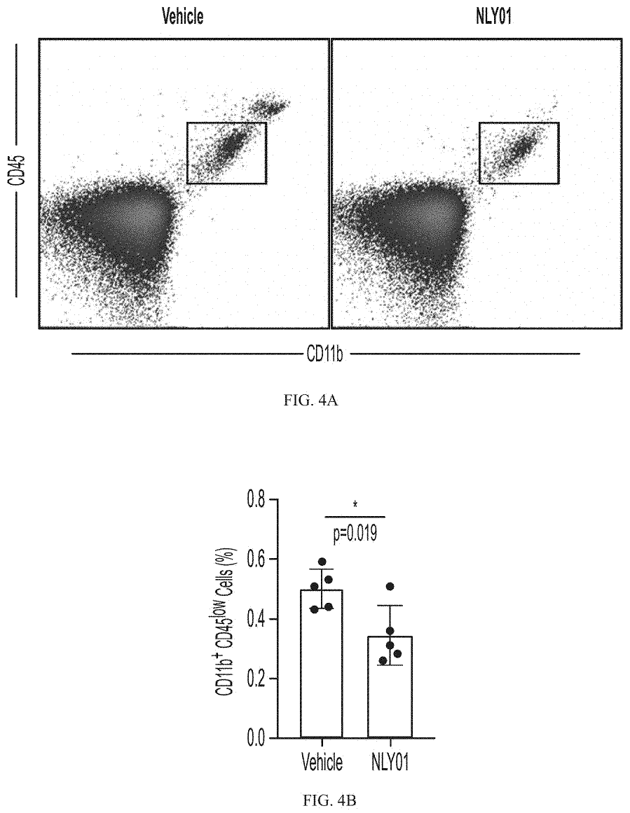

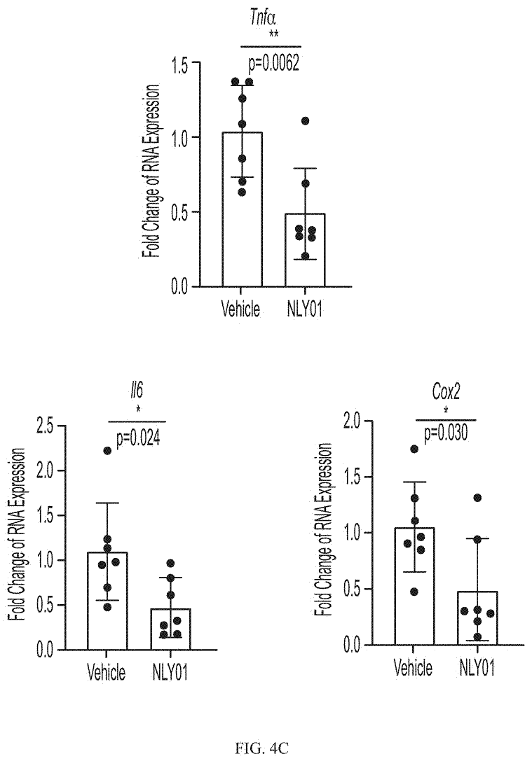

NLY01 modulates retinal microglia proliferation and activation in OIR retina. Microglia activation involves at different stages of DR (8). Activated microglia undergo proliferation and secret multiple inflammatory cytokines, contributing to the development and progression of DR (9). It was further investigated whether NLY01 could regulate microglia in OIR retina. Retinas were collected at P15 and any number changes of microglia were analyzed with flow cytometry. The number of microglia, defined by the cells with CD11b⁺CD45^{low} expression, was reduced in NLY01-treated retinas comparing with vehicle-treated retinas at P15 (FIG. 4A, B), providing evidence that NLY01-treatment reduces the proliferation of microglia in OIR retina.

To determine whether NLY01 could directly regulate microglia activation in retina, the microglia-specific translatomes were retrieved from NLY01-treated or vehicle-treated Cx3cr1^{CreER}:Rp122^{HA} mice with the RiboTag strategy (10). Since this strategy only isolates mRNA associated with HA-epitope-tagged host cell ribosomes, it excludes such exogenous material and hence allows identification of bona fide microglial mRNAs (10). Similar to the whole retina, microglia from NLY01-treated retinas were found to express lower pro-inflammatory cytokines TNFα and IL-6 than those from vehicle-treated retinas (FIG. 4C). In addition, significant reduction of Cox2 expression was found in microglia from NLY01-treated retina. Given microglia are not the only resource of Cox2 in retina, astrocytes also express high amount of Cox2 under the pathological condition, which may explain why the observed decrease in Cox2 expression in whole retina (FIG. 4C).

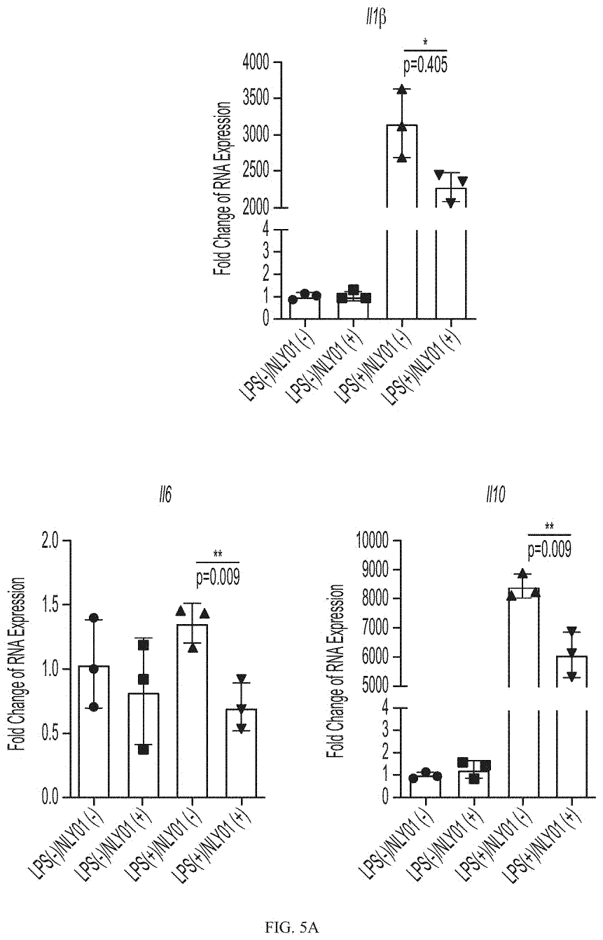

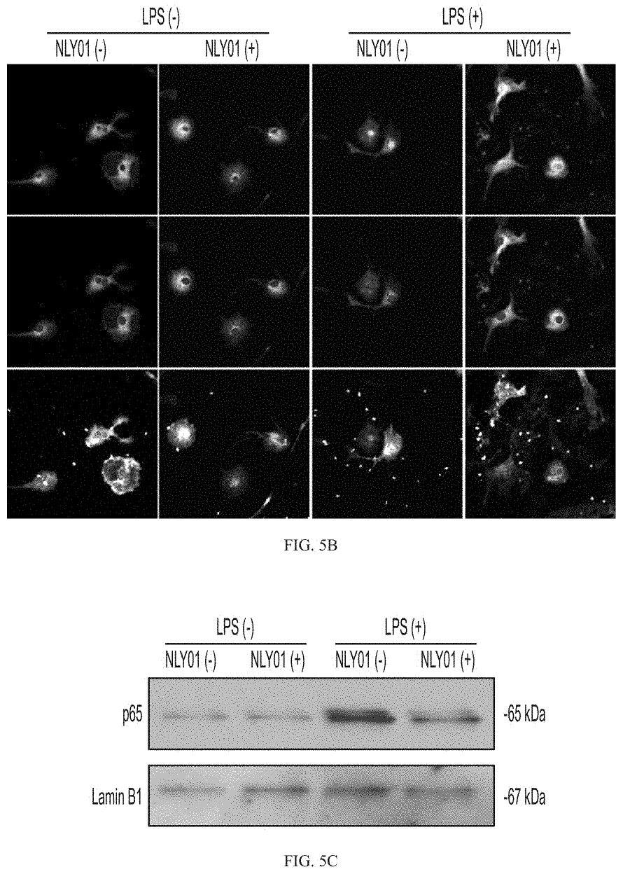

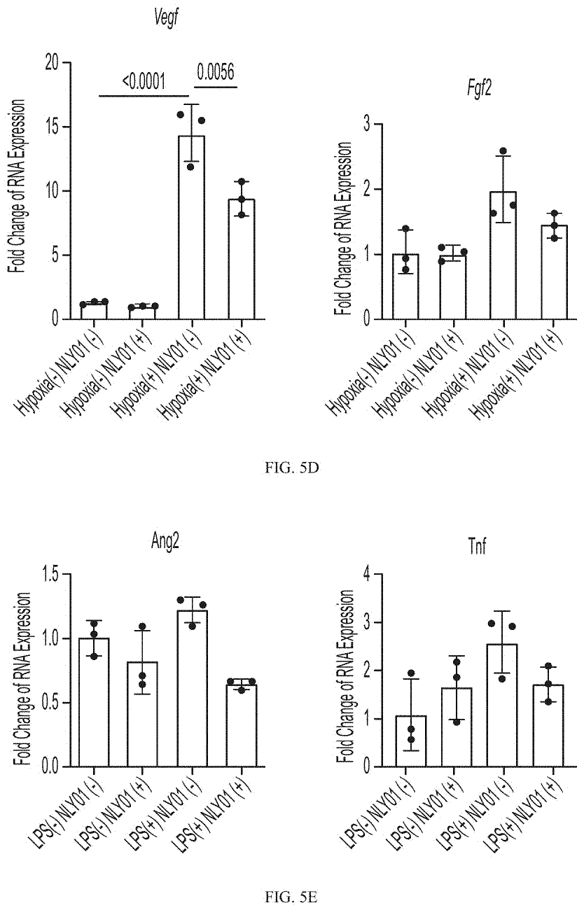

NLY01 ameliorates microglia activation and inhibits NF-kB p65 nuclear translocation in primary microglia. The possible mechanism involved in the microglial regulation of NLY01 was further investigated with primary brain microglia, which can be readily isolated and behave very similarly to retinal microglia, as the retina is an extension of the central nervous system. To study the modulation of brain microglia, both inflammatory and ischemic conditions were studied. First, the expression of inflammatory cytokines in LPS-stimulated (100 ng/mL for 24 h) primary brain microglia with or without NLY01 treatment (1 μM) was examined, with LPS used as an inflammatory stimulator. The NLY01 pretreatment reduced the expression of inflammatory cytokines IL-1(3 and IL-6 (FIG. 5A). The effect of NLY01 was examined on the activation of transcription factor NF-kB, a major transcription factor that promotes inflammation. LPS treatment strongly induced the nuclear translocation of NF-kB p65 in primary brain microglia. With NLY01 pretreatment, there was less nuclear localization of NF-kB p65, indicating suppression of NF-kB activation (FIG. 5B). Consistent with immunostaining, Western blot analysis using nuclear extracts also showed reduced NF-kB p65 translocation into nucleus in the NLY01-pretreatment group (FIG. 5C).

For hypoxic studies, brain microglia were exposed to 1% oxygen and compared with microglia exposed to normoxia (room air) for 24 hours. Cells were pre-treated with NLY01 (1 μM) or vehicle immediately before exposure to hypoxia. Hypoxia increased microglia expression of the pro-angiogenic cytokines VEGF and FGF-2. NLY01 treatment significantly suppressed the hypoxia-induction (FIG. 5D).

Finally, retinal microglia were studied following exposure to LPS. NLY01 pre-treatment downregulated retinal microglial expression of angiopoietin 2 (Ang2) and TNF-α under LPS treatment compared to vehicle (FIG. 5E).

Discussion Relating to NLY01 and OIR and Microglial Modulation

In this current study it was demonstrated that administration of NLY01, a long-acting GLP-1R agonist, promoted physiological re-vascularization, reduced retinal ischemia, and ameliorated pathological angiogenesis in the OIR model, a mice model of ischemic retinopathies including diabetic retinopathy. By regulating microglia activation and modulating neuroinflammation in retina, NLY01 is a potential therapeutic strategy for proliferative diabetic retinopathy and other ischemia-related and neuroinflammatory retinal disease conditions.

Inflammation is a critical contributor for the development of ischemic retinopathies including diabetic retinopathy as well as chronic eye diseases including AMD and glaucoma. A variety of physiologic and molecular abnormalities that are consistent with inflammation have been found to be increased in the retinas or vitreous humor of diabetic animals and patients. Microglia, the resident immune cell in the retina, are closely involved in ocular inflammation. In recent years, numerous studies have shown that microglia play a significant role in ischemic retinopathies including DR, as well as AMD and glaucoma. In the current study, it was demonstrated that NLY01 mainly localized in microglia and reduced the expression of multiple inflammatory genes in OIR retina. To determine whether NLY01 directly regulates microglia in retina, RNA was isolated from HA-tagged ribosome in the microglia/macrophages of NLY01-treated retina of CX3CR₁Cre Ribotag mice. Quantitative real-time PCR studies demonstrated decreased microglia-specific expression of multiple inflammatory genes compares to the vehicle-treated retina (FIG. 4C). These results are consistent with a previous study which found that NLY01 could modulate microglia activation and ameliorate the pathology in the Parkinson's disease (7).

To further investigate the mechanism involved, it was tested whether NLY01 could directly regulate primary cultured microglia. Both inflammatory and ischemic conditions were studied, as these regulate the microglial phenotype in retinal diseases leading to pathogenic, reactive microglia in multiple ocular diseases. In cultured brain microglia, NLY01 inhibited the activation of NF-κB, a master regulator in inflammation. It was found that NLY01 reduced the NF-κB p65 nuclear translocation in the LPS-stimulated primary microglia (FIG. 5B-C), and the expression of several downstream proinflammatory cytokines, providing evidence that NLY01 suppresses inflammation through the regulation of NF-κB. Given its ability to target and modulate reactive microglia, NLY01 is a promising treatment for ischemic retinopathies including diabetic retinopathy, as well as other conditions in which reactive microglia play a pathogenic role, including AMD and glaucoma. In addition, NLY01 downregulated LPS-induction of brain microglial expression of inflammatory cytokines (FIG. 5A) and LPS-induction of retinal microglial expression of pro-angiogenic and pro-inflammatory cytokines (FIG. 5E). Finally, NLY01 modulated hypoxic-induction of brain microglial expression of genes known to promote pathologic angiogenesis (FIG. 5D). Hypoxia is an important stimulator, constituting a critical stimulus in ischemic environments where compromise of blood flow deprives the tissue of oxygen. Together, this indicates the ability of NLY01 to directly modulate reactive microglia under both inflammatory and ischemic conditions and revert them to a more quiescent phenotype, thereby reducing their pathogenic effect in multiple ocular diseases.

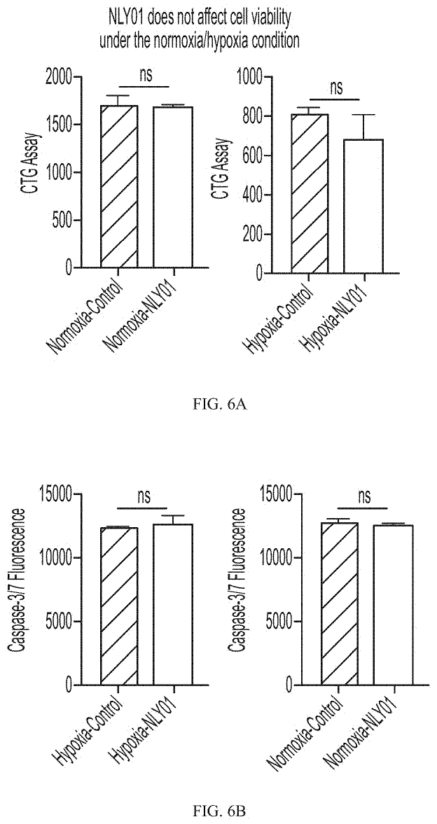

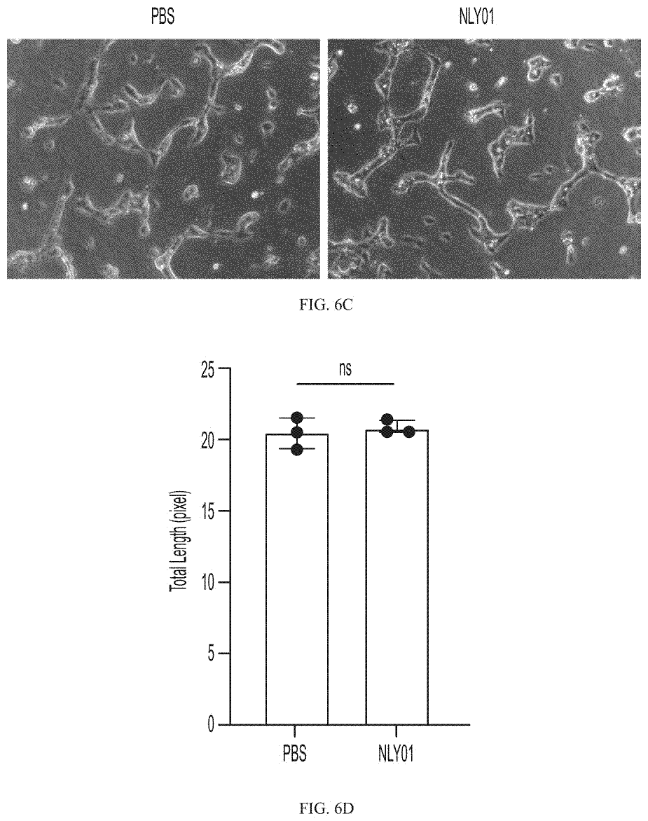

It was also investigated whether NLY01 has any direct effect on angiogenesis in human retinal endothelial cells (HRECs). It was found that NLY01 did not affect apoptosis and proliferation in HRECs. In addition, there was no difference on tube formation between NLY01- and vehicle-treated HRECs, providing evidence that NLY01 does not have any direct effect on HRECs angiogenesis. Up until the current study, there was no information available on the direct angiogenic effect of GLP-1RAs in retinal vascular cells. The current study demonstrates for the first time, that GLP-1RA NLY01 did not exhibit any angiogenic effect in retinal vascular cells (FIG. 6A-D). The vascular protective effect we observed in retina by NLY01 were induced by modulating retinal microglia, rather than retinal endothelial cells.

### Example 2: Laser-Induced Mouse Model of Choroidal Neovascularization (CNV)

The laser-induced CNV model is widely used as a model for wet AMD, in which choroidal neovascularization is the hallmark. The effect of NLY01 was therefore investigated in the mouse laser-induced CNV model.

### Methods: Laser-Induced Choroidal NV and NLY01 Treatment

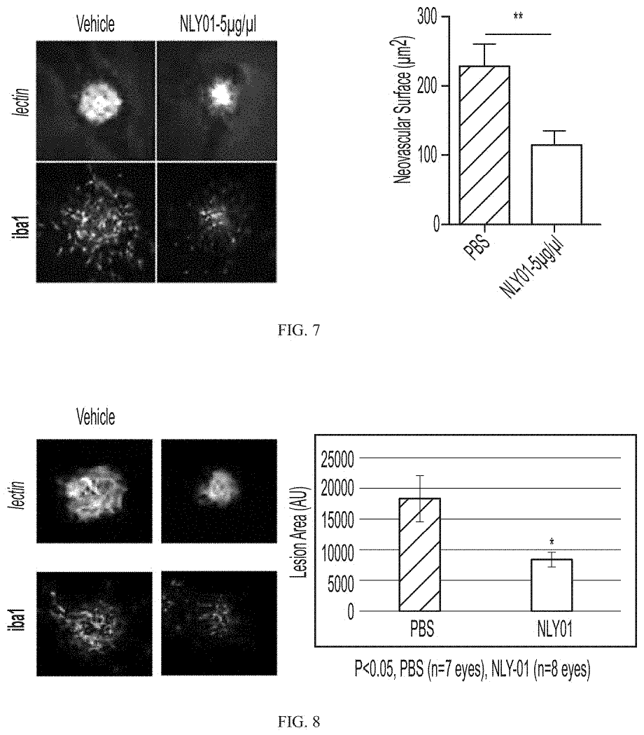

The laser-induced choroidal neovascularization model (laser-induced CNV) is a wide4ly used model of neovascular (wet) age-related macular degeneration (AMD). Experimentally, laser is applied to rupture Bruch membrane beneath the retinal pigment epithelium (RPE) layer in the mouse (or other animal) retina. This leads to a neovascular response of the choriocapillaris (capillaries in the choroid) through the rupture, or CNV. This is the most widely used model for wet AMD. Results are shown in FIGS. 7 and 8.

8-week-old C57BL/6 male mice were subjected to laser-induced rupture of Bruch's membrane at three locations in each eye as previously described (Tobe T et al., (1998) Am J Pathol 153:1641-1646). Briefly, mice were anesthesized and pupils were dilated with 1% tropicamide. Diode laser (532 nm wavelength, 75 μm spot size, 0.1 s duration and 120 mW) with a slit lamp delivery system (OcuLight GL, Iridex, Mountain View, Calif., USA) using a handheld cover slip as a contact lens to view the retina. Production of a bubble at the time of laser, which indicates rupture of Bruch's membrane, is an important factor in obtaining choroidal NV, and only burns in which a bubble was produced were included in the study. For intravitreal treatment, after rupture of Bruch's membrane, mice received 5 μg NLY01 or PBS with intravitreal injection. Choroid was collected 7 days after injection and stained with IB4 to visualize choroidal neovascularization. For subcutaneous treatment, NLY01 (10 mg/kg) was injected subcutaneously right after laser burn, and again 3 days later. Choroid was collected 7 days after laser to visualize CNV.

### Discussion Relating to NLY01 and Laser-Induced CNV

Intravitreal NLY01 treatment resulted in a significant reduction in choroidal neovascularization compared to control eyes that received PBS injection (FIG. 7). In addition, in a separate experiment, subcutaneous administration of NLY01 significantly inhibited laser-induced CNV as compared to PBS treatment (FIG. 8). These results provide evidence that both local (for instance, intravitreal) and systemic (for instance, subcutaneous) NLY01 treatment could be used for patients with wet AMD, similar to current intravitreal anti-VEGF treatments.

### Example 3: Retinal Degeneration

Retinal neurodegeneration is an important element in multiple retinal conditions, including inherited retinal degenerations and dry age-related macular degeneration, characterized by degeneration of photoreceptors, as well as glaucoma, characterized by death and degeneration of retinal ganglion cells. A commonly used model for studying retinal neurodegeneration is the rd10 retinal degeneration mouse model, which originates from mice in which there is a mutation in the phosphodiesterase 6 gene (PDE6 mutation) that has been found in some patients with retinitis pigmentosa. These mice develop gradual death of the photoreceptors, and activated microglia are thought to play a role in hastening this photoreceptor cell death. The model is therefore one of the most widely used models of hereditary retinal degenerations including retinitis pigmentosa. This model also has implications for non-neovascular (dry) AMD, since photoreceptor death is a critical component of dry AMD, as well as glaucoma, in which reactive microglia contribute to retinal ganglion cell death.

Methods Relating to Rd10 and NLY01 Treatment

B6.CXB1-Pde6βrd10/J (rd10) mice were purchased from the Jackson Laboratory. For intravitreal injection group, mice received 5 μg NLY01 at P7, P14, P21, P28. The contralateral eye were injected with PBS as control. Electroretinography (ERG) was performed at P30 to evaluate the protective effect of NLY01. For intraperitoneal injection, mice were randomly dived into 3 groups which received 3 mg/kg, 10 mg/kg or PBS. Mice received NLY01 or PBS every other day from P14-P34, ERGs were performed at P35.

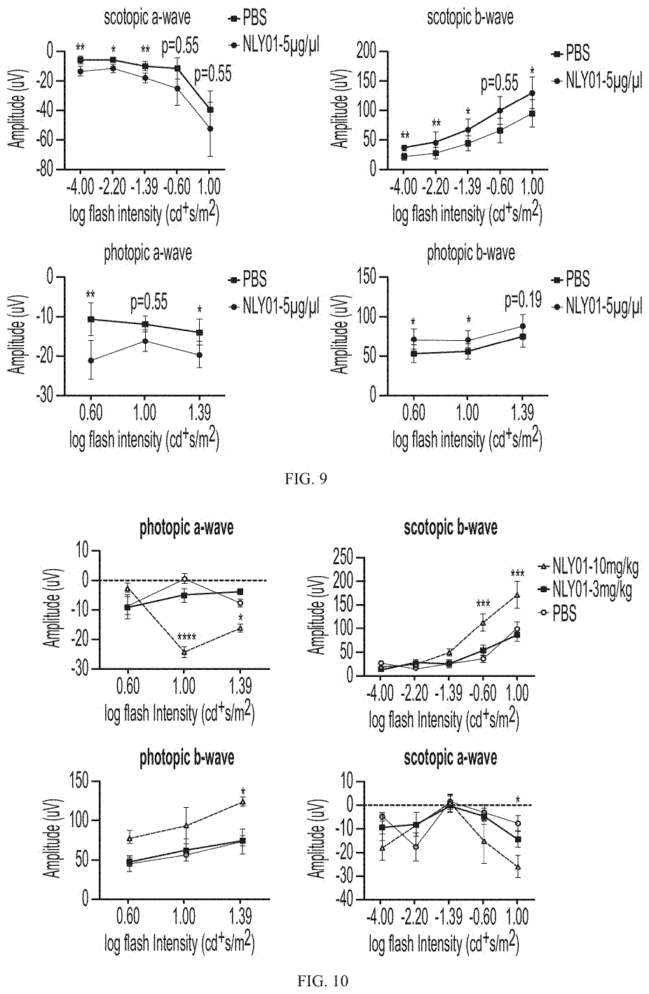

The results are shown in FIGS. 9 and 10. A quantitative approach for determining if a candidate therapy can be protective against photoreceptor degeneration in the rd10 model is to perform electroretinography studies (ERG), which demonstrates the electrical activity of the retina. The a-wave shows photoreceptor activity, while the b-wave shows the inner retinal activity, downstream of photoreceptor activity. ERGs are a useful way to determine neuroretinal function. Scotopic ERG highlights rod photoreceptor activity, while photopic ERG highlights cone photoreceptor activity.

As shown in FIG. 9, for both the scotopic α-wave and b-wave and the photopic α-wave and b-wave, NLY01 treatment significantly improved the amplitudes at the indicated flash intensities (denoted by *). This indicates that intravitreal injection of NLY01 improved both outer retinal (photoreceptor) function and inner retinal function.

FIG. 10 shows the results from intraperitoneal injection of NLY01 (at either 10 mg/kg or 3 mg/kg) or PBS vehicle every 2 days. As shown in FIG. 10, for both the scotopic α-wave and b-wave and the photopic α-wave and b-wave, NLY01 treatment showed a dose-dependent response. At the higher dose of 10 mg/kg, NLY01 significantly improved the amplitudes at the indicated flash intensities (denoted by *). This indicates that systemic (intraperitoneal) administration of NLY01 improved both outer retinal (photoreceptor) function and inner retinal function.

### Discussion Relating to NLY01 and Retinal Degeneration

These experiments provide evidence that both intravitreal and intraperitoneal NLY01 treatment could be used for neuroprotection for patients with photoreceptor degenerations, including hereditary retinal degenerations and AMD. Since there are shared mechanisms in photoreceptor degeneration in dry AMD, this could indicate the benefit of NLY01 for dry AMD as well. In addition, these studies demonstrate that NLY01 could offer neuroprotection for other ocular conditions including glaucoma, in which reactive microglia play a pathogenic role in retinal ganglion cell neurodegeneration.

### Example 4: Retinal Ganglion Cell Death

Retinal ganglion cell death underlies major causes of blindness from both chronic conditions such as glaucoma and acute conditions such as acute glaucoma, ischemic optic neuropathy, and optic neuritis. Retinal ganglion cells with their axonal projections comprise the optic nerve connecting the retina to the brain. The large group of diseases that include retinal ganglion cell death are known as optic neuropathies. An important model for studying retinal ganglion cell degeneration is the retinal ischemia-reperfusion (I/R) model, an experimental model in which the intraocular pressure is raised to stop retinal blood flow (ischemia) and then allowed to normalize to restore blood flow (reperfusion). This reperfusion is associated by both an increase in oxidative stress and pro-inflammatory processes, resulting in death of retinal neuronal elements, especially retinal ganglion cells (RGCs). This model has been long been used to study neuroprotective therapies of retinal ganglion cells (M Hangai et al., Exp Eye Research 1996; 63, 501-509; AH Neufeld et al., Exp Eye Research 2002; 75:521-8). Given the pathophysiologic contribution of inflammation and oxidative stress to the RGC loss and dysfunction, the I/R model has been useful for identifying therapies for optic neuropathies like glaucoma as well as ischemic retinopathies including diabetic retinopathy.

Methods Relating to Retinal Ischemia-Reperfusion and NLY01 Treatment

Retinal ischemia-reperfusion was induced in mice as previously described (M Hartsock et al., JoVE 2016; 113:54065). Intraocular pressure was raised to 110 mm Hg for 90 minutes to halt retinal blood flow. IOP was then allowed to normalize to restore retinal perfusion. Mice received either vehicle or NLY01 (3 mg/kg via subcutaneous injection) one day before I/R and one day after I/R. Mice were sacrificed 5 days after I/R, and retina whole-mounts prepared for immunostaining with the neuronal cell marker NeuN as previously described (Z Xu et al., J Neurochemistry 2015; 133:233-241). Survival of neuronal cells in the ganglion cell layer was analyzed and quantitated using confocal Zeiss microscopy.

For each retina, three random fields in mid-peripheral retina were used for image taking. NeuN-positive cells in GCL (ganglion cell layer) were then counted using the Image J program (NIH).

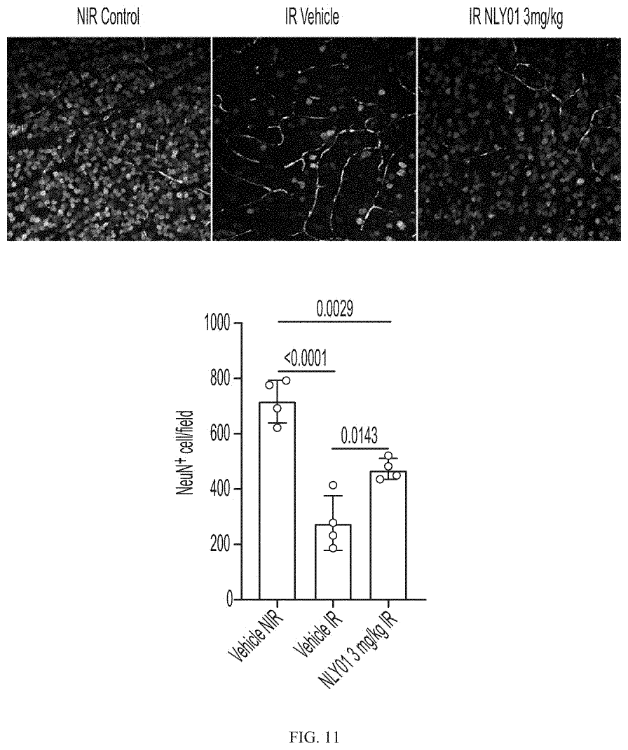

The results are shown in FIG. 11. Retinal I/R resulted in significant reduction in retinal ganglion cell counts as demonstrated by NeuN staining as compared to no I/R, as expected. Compared to I/R eyes in mice receiving vehicle treatment, NLY01 treatment resulted in a significant increase in retinal ganglion cell counts following retinal I/R, indicating a strong neuroprotective effect of NLY01.

### Discussion Relating to NLY01 and Retinal Ganglion Cell Loss

This experiment provides evidence that NLY01 treatment by systemic administration can be used for neuroprotection for diseases characterized by retinal ganglion cell loss, including glaucoma and other optic neuropathies. In addition, since retinal diseases including nonproliferative diabetic retinopathy are also characterized by neuronal and ganglion cell loss caused by oxidative stress and pro-inflammatory processes, this experiment provides further evidence of the usefulness of NLY01 for DR.

Together, these results demonstrate that NLY01 treatment could be beneficial several models of retinal disease, including diabetic retinopathy, diabetic macular edema, wet AMD, hereditary retinal degenerations, dry AMD, and glaucoma and other optic neuropathies.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A method of treating an ocular neuroinflammatory disease, an ocular disease or disorder, an ocular autoimmune disease or ocular injury comprising:
administering to a subject a therapeutically effective amount of at least one glucagon-like peptide 1 receptor (GLP-1r) agonist,
thereby treating an ocular neuroinflammatory disease, an ocular disease or disorder, an ocular autoimmune disease or ocular injury.

2. The method of claim 1 comprising administering to a subject in need of an effective amount of GLP-1r agonist to block activation of microglia in the eye.

3. The method of claim 1, wherein a GLP-1r agonist comprises: PEGylated exenatide (NLY01), exenatide, liraglutide, exenatide LAR (long acting), taspoglutide, albiglutide, dulaglutide, lixisenatide, semaglutide, LY2189265 or combinations thereof.

4. The method of claim 1, wherein the GLP-1r agonist is PEGylated exenatide (NLY01).

5. The method of claim 1, wherein the GLP-1r agonist is administered systemically or locally.

6. The method of claim 1, wherein the administration of the GLP-1r agonist composition comprises subretinal injection, intravitreal injection, subconjunctiva injection, or intraocular injection.

7. The method of claim 1, wherein the ocular neuroinflammatory disease, the ocular disease or disorder, the ocular autoimmune disease or ocular injury comprises: neuromyelitis optica spectrum disorder, optic neuritis, acute disseminated encephalomyelitis (ADEM), autoimmune uveitis, intraocular inflammation, uveitis, uveoretinitis, proliferative vitreoretinopathy, proliferative diabetic retinopathy (PDR), macular edema, non-proliferative diabetic retinopathy, dry and wet age-related macular degeneration, glaucoma, optic neuropathies, branch and central retinal vein occlusion, ocular neovascularization, retinopathy of prematurity (ROP), a retinal vein occlusion, ocular ischemic syndrome, neovascular glaucoma, a retinal hemangioma, Coates' disease, familial exudative vitreoretinopathy (FEVR), Norrie disease, macular degeneration, diabetic retinopathy, retinitis pigmentosa, cone dystrophy geographic atrophy, detachment ischemia, optic nerve neuritis, ocular cancer, glaucoma, retinal trauma, physical trauma to the optic nerve and surrounding tissues, or retinal nerve damage.

8-17. (canceled)

18. A method of treating an ocular neuroinflammatory disease, an ocular disease or disorder, an ocular autoimmune disease or ocular injury comprising:
administering to a subject a therapeutically effective amount of long-acting PEGylated exenatide (NLY01), exenatide, liraglutide, exenatide LAR (long acting), taspoglutide, albiglutide, dulaglutide, lixisenatide, or semaglutide, LY2189265.

19-20. (canceled)

21. A kit comprising:
(a) one or more GLP-1r agonists;
(b) written instructions for use of the one or more GLP-1r agonists for treating an ocular neuroinflammatory disease, an ocular disease or disorder, an ocular autoimmune disease or ocular injury.

22. (canceled)

23. The method of claim 1 wherein the subject is identified as suffering from an ocular neuroinflammatory disease and PEGylated exenatide (NLY01), exenatide, liraglutide, exenatide LAR (long acting), taspoglutide, albiglutide, dulaglutide, lixisenatide, semaglutide, LY2189265 or combinations thereof are administered to the identified subject.

24. The method of claim 1 wherein the subject is identified as suffering from an ocular disease or disorder and PEGylated exenatide (NLY01), exenatide, liraglutide, exenatide LAR (long acting), taspoglutide, albiglutide, dulaglutide, lixisenatide, semaglutide, LY2189265 or combinations thereof are administered to the identified subject.

25. The method of claim 1 wherein the subject is identified as suffering from an ocular autoimmune disease and PEGylated exenatide (NLY01), exenatide, liraglutide, exenatide LAR (long acting), taspoglutide, albiglutide, dulaglutide, lixisenatide, semaglutide, LY2189265 or combinations thereof are administered to the identified subject.

26. The method of claim 1 wherein the subject is identified as suffering from an ocular injury and PEGylated exenatide (NLY01), exenatide, liraglutide, exenatide LAR (long acting), taspoglutide, albiglutide, dulaglutide, lixisenatide, semaglutide, LY2189265 or combinations thereof are administered to the identified subject.
